(19) United States
(12) Patent Application Publication
(10) Pub. No.: US 2026/0020986 A1
(43) Pub. Date: Jan. 22, 2026

(54) MEDICATED DRESSINGS

(71) Applicant: RAMBAM MEDTECH LTD., Haifa (IL)

(72) Inventors: Benjamin MEDALION, Zikron Yakov (IL); Yaron SHRIM, Mevaseret Zion (IL); Itzhak AHARON, Jerusalem (IL)

(73) Assignee: RAMBAM MEDTECH LTD., Haifa (IL)

(21) Appl. No.: 18/994,966

(22) PCT Filed: Jun. 15, 2023
(86) PCT No.: PCT/IL2023/050619

§ 371 Date: Jan. 15, 2025

Related U.S. Application Data
(60) Provisional application No. 63/391,134, filed on Jul. 21, 2022.
(60) Provisional application No. 63/417,390, filed on Oct. 19, 2022.

(51) Int. Cl. A61F 13/02; A61F 13/00; A61M 25/02; A61M 39/02

(52) U.S. Cl. CPC A61F 13/02; A61M 25/02; A61M 39/0247; A61F 2013/00412; A61L 2300/404; A61M 2025/0266

(57) ABSTRACT

A dressing is provided that includes an upper layer, which defines an insertion area having a non-adhesive skin-facing surface configured to be positioned above a skin-insertion site of a patient, and is configured to allow passage of a percutaneous medical device through the insertion area. An adhesive area has a skin-facing surface coated with an adhesive. The insertion area is shaped so as to define a fillable insertion chamber between the non-adhesive skin-facing surface and the skin when the dressing is adhered to the skin by the adhesive area. One or more reservoirs, separate from the fillable insertion chamber, contain a flowable substance, and are connected to the insertion area by one or more passages, so as to enable flow of the flowable substance from the one or more reservoirs to the insertion area upon application of pressure to the one or more reservoirs. Other embodiments are also described.

### CROSS-REFERENCE TO RELATED APPLICATIONS

The present application claims priority from U.S. Provisional Application 63/391,134, filed Jul. 21, 2022, and U.S. Provisional Application 63/417,390, filed Oct. 19, 2022. All of the above-referenced applications are assigned to the assignee of the present application and incorporated herein by reference.

### FIELD OF THE APPLICATION

The present invention relates generally to dressings, and specifically to medicated dressings, including antimicrobial catheter dressings.

### BACKGROUND OF THE APPLICATION

Catheter-related blood-stream infections (CRBSIs), exit-site infections, and tunnel infections are complications related to central vascular catheters (CVC), peripheral vascular catheters, and other catheters, tubes, or drivelines penetrating the skin. In the United States, 15 million CVC days (i.e., the total number of days of exposure to CVCs among all patients in the selected population during the selected time period) occur in intensive care units each year. These infections independently increase hospital costs and length of stay. While 80,000 CRBSIs occur in ICUs each year, a total of 250,000 cases have been estimated to occur annually, if entire hospitals are assessed. The cost of these infections is substantial, both in terms of morbidity and financial resources expended. The role of peripheral IVs as causes of bloodstream infection is likely underestimated. Among hemodialysis CVC use, CRBSIs alone have a reported incidence of 1.1 to 5.5 episodes per 1000 catheter days and are associated with increased morbidity, hospitalization, and death. Approximately one-third of CRBSIs will require treatment in the hospital with significant financial burden. The average cost of each hospitalization is estimated at $22,000 per episode of bacteremia.

Among the recognized routes for catheters contamination are:
Migration of skin organisms at the insertion site into the cutaneous catheter tract and along the surface of the catheter with colonization of the catheter tip, is considered the most common route of infection for short-term catheters.
Direct contamination of the catheter or catheter hub by contact with hands or contaminated fluids or devices is considered another common route of infection.

It is recommended to replace dressings used on short-term CVC sites every 2 days for gauze dressings, and to replace dressings used on short-term CVC sites at least every 7 days for transparent dressings. It is also advised to use a sutureless securement device to reduce the risk of infection for intravascular catheters.

Use of chlorhexidine gluconate-impregnated sponge dressings with intravascular catheters in the intensive care unit reduced risk of infection even when background infection rates were low. Reducing the frequency of changing unsoiled adherent dressings from every 3 days to every 7 days modestly reduces the total number of dressing changes and appears safe.

US Patent Application Publication 2019/0083752 to Howell et al. describes a full-surround contact antimicrobial dressing. The dressing includes a transparent body that covers a skin insertion site through which a catheter assembly passes for disposal within the body of a patient. The transparency of the dressing body enables inspection of the skin insertion site. In one embodiment, therefore, an antimicrobial full-surround contact dressing for use with a medical device inserted into a skin surface of a patient via a skin insertion site is disclosed and comprises a transparent flat body, an antimicrobial adhesive substance disposed on a bottom surface of the body, and a slit defined in the body. The slit is configured to enable the body to be placed fully around a perimeter of the medical device on the skin surface at the skin insertion site such that the bottom surface of the body fully surrounds and contacts the skin insertion site.

U.S. Pat. No. 7,662,159 to Brandigi describes a transcutaneous access shield with an instrument opening and surrounding wall to prevent infection during surgical and other medical procedures. The shield resists the entry of microbes from the skin at the body access point. A shield positioner and skin hook can be used to improve placement of the shield.

US Patent Application Publication 2007/0093760 to Wexler describes a syringe including a wound dressing for arresting bleeding and/or providing a barrier against microorganisms at a needle insertion point on the skin of a patient. The wound dressing is releasably integrated with the syringe. In some embodiments, the syringe includes a bandage disposed on the posterior end of the needle hub of the syringe such that the needle extends through the bandage. In other embodiments the syringe includes a reservoir containing a dressing substance. The dressing may be adhered, or otherwise applied, to the skin of the patient proximate the needle insertion point prior to withdrawing the needle of the syringe from the patient. As the needle is withdrawn, the dressing remains at the insertion point on the skin of the patient to arrest bleeding and/or provide a barrier against microorganisms at the wound site.

US Patent Application Publication 2018/0161543 to Burkholz describes catheter securement dressings that aid in applying skin adhesive to a catheter insertion site. Catheter securement dressings can include a first dressing layer that secures a position of a catheter at a catheter insertion site following catheterization. The first dressing layer can include an access window that provides access to the catheter insertion site to allow for application of skin adhesive to the catheter insertion site. After the skin adhesive has dried, a second dressing layer can be folded over the first dressing layer to cover the access window.

### SUMMARY OF THE APPLICATION

Some embodiments of the present invention provide a dressing for use with a percutaneous medical device, such as a catheter, for insertion through skin of a patient at a skin-insertion site. The dressing defines an insertion area having a non-adhesive skin-facing surface configured to be positioned above the skin-insertion site, and a skin-facing adhesive area disposed around at least a portion of the insertion area. When the dressing is adhered to the skin, the insertion area is shaped so as to define a fillable insertion chamber between the non-adhesive skin-facing surface and the skin.

The dressing further comprises a flowable substance, such as an antimicrobial agent, contained in one or more reservoirs of the dressing. The one or more reservoirs are separate from the fillable insertion chamber, and are connected to the fillable insertion chamber by one or more passages of the dressing.

After passage of the percutaneous medical device through the insertion area and the skin-insertion site, application of pressure to the one or more reservoirs causes flow of the flowable substance from the one or more reservoirs to the insertion area via the one or more passages.

The flowable substance generally floods the fillable insertion chamber, thereby coating both the skin-insertion site and a relatively long portion of the percutaneous medical device that passes through the fillable insertion chamber of the dressing. This coating of the external portion of the percutaneous medical device generally inhibits bacteria migration along the instrument, which is believed to be a common cause of skin-insertion site infections. The dressing thus generally provides better and longer-term infection protection at the skin-insertion site than conventional dressings. As a result, the dressing generally reduces the risk of site infection and the required frequency of dressing changes.

The percutaneous medical device may comprise any medical device that is disposed partially within the body and partially exposed outside the body, and passes through a disruption in the skin. For example, the percutaneous medical device may comprise a percutaneous-insertion instrument, such as a catheter, or an orthopedic percutaneous fixation device, such as a percutaneous pin.

There is therefore provided, in accordance with an Inventive Concept 1 of the present invention, a dressing for use with a percutaneous medical device for insertion through skin of a patient at a skin-insertion site, the dressing comprising:
an upper layer, which (a) defines an insertion area having a non-adhesive skin-facing surface configured to be positioned above the skin-insertion site, (b) is shaped so as to define a slit having first and second edges, and (c) is configured to allow passage of the percutaneous medical device through the insertion area via an opening through the insertion area defined at a junction of respective inner ends of the first and the second edges of the slit, respectively; and
an adhesive area, which has a skin-facing surface coated with an adhesive, and which includes (a) a peripheral portion disposed around at least a portion of a perimeter of the insertion area, and (b) an adhesive-area slit-closing portion disposed alongside at least the first edge of the slit and configured to be adhered to an upper surface of an upper-layer slit-closing portion of the upper layer alongside the second edge of the slit,
wherein the insertion area is shaped so as to define a fillable insertion chamber between the non-adhesive skin-facing surface and the skin when (a) the dressing is adhered to the skin by at least the peripheral portion of the adhesive area and (b) the adhesive-area slit-closing portion of the adhesive area is adhered to the upper surface of the upper-layer slit-closing portion of the upper layer.
Inventive Concept 2. The dressing according to Inventive Concept 1, wherein the slit is wedge-shaped.
Inventive Concept 3. The dressing according to Inventive Concept 1,
   wherein a percutaneous-medical-device-adhering portion of the adhesive area is disposed on a skin-facing surface of the insertion area at least partially surrounding the opening at the junction, and
   wherein the percutaneous-medical-device-adhering portion is configured to adhere to an external surface of the percutaneous medical device when the percutaneous medical device is disposed passing through the opening.
Inventive Concept 4. The dressing according to Inventive Concept 1, wherein the insertion area of the upper layer is flexible.
Inventive Concept 5. The dressing according to Inventive Concept 1, wherein the insertion area of the upper layer is elastic.
Inventive Concept 6. The dressing according to any one of Inventive Concepts 1-5, wherein the dressing is shaped such that adhering of the adhesive-area slit-closing portion of the adhesive area to the upper surface of the upper-layer slit-closing portion of the upper layer forms the fillable insertion chamber.
Inventive Concept 7. The dressing according to Inventive Concept 6, wherein a skin-facing surface of the upper-layer slit-closing portion of the upper layer is non-adhesive, such that the upper-layer slit-closing portion of the upper layer can move away from the skin during formation of the fillable insertion chamber by application of the adhesive-area slit-closing portion of the adhesive area to the upper surface of the upper-layer slit-closing portion of the upper layer.
Inventive Concept 8. The dressing according to Inventive Concept 6, wherein the dressing is shaped such that adhering of the adhesive-area slit-closing portion of the adhesive area to the upper surface of the upper-layer slit-closing portion of the upper layer provides a tented shape to the fillable insertion chamber.
Inventive Concept 9. The dressing according to Inventive Concept 8, wherein the dressing is shaped such that adhering of the adhesive-area slit-closing portion of the adhesive area to the upper surface of the upper-layer slit-closing portion of the upper layer causes the tented shape to define a single apex.
Inventive Concept 10. The dressing according to any one of Inventive Concepts 1-5,
   wherein the dressing further comprises a seal shaped so as to define a seal slit having first and second seal-slit edges, the seal having properties different in at least one respect from those of material of a remainder of the insertion area,
   wherein the seal is coupled to the insertion area at least partially surrounding the opening at the junction, and
   wherein the seal is configured to form a tight seal around the percutaneous medical device when the percutaneous medical device is passed through the seal and the opening through the insertion area, and the adhesive-area slit-closing portion of the adhesive area is adhered to the upper surface of the upper-layer slit-closing portion of the upper layer alongside the second edge of the slit of the insertion area.
Inventive Concept 11. A kit comprising the dressing according to any one of Inventive Concepts 1-5,
   wherein the kit further comprises a sealing support, which is shaped so as to define (a) an upper surface, (b) a lower surface, and (c) a hole, which (i) passes through the sealing support between the upper and the lower surfaces, and (ii) is configured to form a tight seal around the percutaneous medical device when the percutaneous medical device is disposed passing through the hole.
   wherein a sealing-support-adhering portion of the adhesive area is disposed on a skin-facing surface of the insertion area at least partially surrounding the opening at the junction, and
   wherein the sealing-support-adhering portion is configured to adhere to the upper surface of the sealing support when the percutaneous medical device is disposed passing through the hole of the sealing support and the opening through the insertion area defined by the upper layer of the dressing.
Inventive Concept 12. A kit comprising the dressing according to any one of Inventive Concepts 1-5, the kit further comprising the percutaneous medical device,
   wherein the percutaneous medical device comprises a sealing support, which is shaped so as to define an upper surface and a lower surface, wherein the percutaneous medical device is disposed passing through the sealing support such that the sealing support surrounds a portion of the percutaneous medical device,
   wherein a sealing-support-adhering portion of the adhesive area is disposed on a skin-facing surface of the insertion area at least partially surrounding the opening at the junction, and
   wherein the sealing-support-adhering portion is configured to adhere to the upper surface of the sealing support when the percutaneous medical device is disposed passing through the opening through the insertion area defined by the upper layer of the dressing.
Inventive Concept 13. The dressing according to any one of Inventive Concepts 1-5, further comprising:
   a flowable substance;
   one or more reservoirs, which are separate from the fillable insertion chamber, and contain the flowable substance; and
   one or more passages connecting the one or more reservoirs of the flowable substance and the insertion area so as to enable flow of the flowable substance from the one or more reservoirs to the insertion area upon application of pressure to the one or more reservoirs.
Inventive Concept 14. The dressing according to any one of Inventive Concepts 1-5, wherein the percutaneous medical device is a percutaneous-insertion instrument, and wherein the upper layer is configured to allow passage of the percutaneous-insertion instrument through the insertion area via the opening.
Inventive Concept 15. The dressing according to Inventive Concept 14, wherein the percutaneous-insertion instrument is a tube, and wherein the upper layer is configured to allow passage of the tube through the insertion area via the opening.
Inventive Concept 16. The dressing according to Inventive Concept 15, wherein the tube is selected from the group of tubes consisting of: a catheter and a cannula, and wherein the upper layer is configured to allow passage of the selected tube through the insertion area via the opening.
Inventive Concept 17. The dressing according to Inventive Concept 14, wherein the percutaneous-insertion instrument is selected from the group of instruments consisting of: a cable and a wire, and wherein the upper layer is configured to allow passage of the selected instrument through the insertion area via the opening.
Inventive Concept 18. The dressing according to any one of Inventive Concepts 1-5, wherein the percutaneous medical device is an orthopedic percutaneous fixation device, and wherein the upper layer is configured to allow passage of the orthopedic percutaneous fixation device through the insertion area via the opening.

There is further provided, in accordance with an Inventive Concept 19 of the present invention, a method comprising:
applying a dressing to skin of a patient by:
   positioning, above a skin-insertion site, a non-adhesive skin-facing surface of an insertion area of an upper layer of the dressing, wherein the insertion area of the upper layer is shaped so as to define a slit having first and second edges, and
   adhering at least a skin-facing surface of a peripheral portion of an adhesive area of the dressing to the skin, wherein the peripheral portion of the adhesive area is disposed around at least a portion of a perimeter of the insertion area and is coated with an adhesive;
adhering an adhesive-area slit-closing portion of the adhesive area disposed alongside at least the first edge of the slit to an upper surface of an upper-layer slit-closing portion of the upper layer alongside the second edge of the slit, such that the insertion area is shaped so as to define a fillable insertion chamber between the non-adhesive skin-facing surface and the skin;
causing a percutaneous medical device to pass through the insertion area via an opening through the insertion area defined at a junction of respective inner ends of the first and the second edges of the slit, respectively; and
before or after causing the percutaneous medical device to pass through the insertion area, passing the percutaneous medical device through the skin at the skin-insertion site.
Inventive Concept 20. The method according to Inventive Concept 19, wherein causing the percutaneous medical device to pass through the insertion area via the opening comprises, after passing the percutaneous medical device through the skin at the skin-insertion site, positioning the first and the second edges of the slit around a longitudinal portion of the percutaneous medical device such that the percutaneous medical device passes through the opening.
Inventive Concept 21. The method according to Inventive Concept 19, wherein adhering the adhesive-area slit-closing portion of the adhesive area to the upper surface of the upper-layer slit-closing portion of the upper layer provides a tented shape to the fillable insertion chamber.
Inventive Concept 22. The method according to Inventive Concept 19, wherein the slit is wedge-shaped.
Inventive Concept 23. The method according to Inventive Concept 19,
   wherein a skin-facing surface of the upper-layer slit-closing portion of the upper layer is non-adhesive, and
   wherein adhering the adhesive-area slit-closing portion of the adhesive area to the upper surface of the upper-layer slit-closing portion of the upper layer causes the upper-layer slit-closing portion of the upper layer to move away from the skin during formation of the fillable insertion chamber.
Inventive Concept 24. The method according to Inventive Concept 19, wherein adhering the adhesive-area slit-closing portion of the adhesive area to the upper surface of the upper-layer slit-closing portion of the upper layer provides a tented shape to the fillable insertion chamber.
Inventive Concept 25. The method according to Inventive Concept 24, wherein adhering the adhesive-area slit-closing portion of the adhesive area to the upper surface of the upper-layer slit-closing portion of the upper layer causes the tented shape to define a single apex.
Inventive Concept 26. The method according to Inventive Concept 19,
   wherein a percutaneous-medical-device-adhering portion of the adhesive area is disposed on a skin-facing surface of the insertion area at least partially surrounding the opening at the junction, and
   wherein causing the percutaneous medical device to pass through the insertion area via the opening comprises adhering the percutaneous-medical-device-adhering portion to an external surface of the percutaneous medical device.
Inventive Concept 27. The method according to Inventive Concept 19, wherein the insertion area of the upper layer is flexible.
Inventive Concept 28. The method according to Inventive Concept 19, wherein the insertion area of the upper layer is elastic.
Inventive Concept 29. The method according to Inventive Concept 19, wherein causing the percutaneous medical device to pass through the insertion area comprises causing a percutaneous-insertion instrument to pass through the insertion area via the opening.
Inventive Concept 30. The method according to Inventive Concept 29, wherein causing the percutaneous medical device to pass through the insertion area comprises causing a tube to pass through the insertion area via the opening.
Inventive Concept 31. The method according to Inventive Concept 30, wherein the tube is selected from the group of tubes consisting of: a catheter and a cannula, and wherein causing the tube to pass through the insertion area comprises causing the selected tube to pass through the insertion area via the opening.
Inventive Concept 32. The method according to Inventive Concept 29, wherein the percutaneous-insertion instrument is selected from the group of instruments consisting of: a cable and a wire, and wherein causing the percutaneous-insertion instrument to pass through the insertion area comprises causing the selected instrument to pass through the insertion area via the opening.
Inventive Concept 33. The method according to Inventive Concept 19, wherein causing the percutaneous medical device to pass through the insertion area comprises causing an orthopedic percutaneous fixation device to pass through the insertion area via the opening.
Inventive Concept 34. The method according to Inventive Concept 19,
   wherein the dressing further includes a seal shaped so as to define a seal slit having first and second seal-slit edges, the seal having properties different in at least one respect from those of material of a remainder of the insertion area,
   wherein the seal is coupled to the insertion area at least partially surrounding the opening at the junction, and
   wherein adhering the adhesive-area slit-closing portion of the adhesive area comprises adhering the adhesive-area slit-closing portion of the adhesive area to the upper surface of the upper-layer slit-closing portion of the upper layer alongside the second edge of the slit such that the seal forms a tight seal around the percutaneous medical device.
Inventive Concept 35. The method according to Inventive Concept 19,
   wherein the method further comprises, before causing the percutaneous medical device to pass through the insertion area, disposing the percutaneous medical device passing through a hole defined by a sealing support such that the hole forms a tight seal around the percutaneous medical device, the hole passing between an upper surface and a lower surface of the sealing support,
   wherein a sealing-support-adhering portion of the adhesive area is disposed on a skin-facing surface of the insertion area at least partially surrounding the opening at the junction, and
   wherein causing the percutaneous medical device to pass through the insertion area via the opening comprises adhering the sealing-support-adhering portion to the upper surface of the sealing support.
Inventive Concept 36. The method according to Inventive Concept 19,
   wherein the percutaneous medical device includes a sealing support, which is shaped so as to define an upper surface and a lower surface, wherein the percutaneous medical device is disposed passing through the sealing support such that the sealing support surrounds a portion of the percutaneous medical device,
   wherein a sealing-support-adhering portion of the adhesive area is disposed on a skin-facing surface of the insertion area at least partially surrounding the opening at the junction, and
   wherein causing the percutaneous medical device to pass through the insertion area via the opening comprises adhering the sealing-support-adhering portion to the upper surface of the sealing support.
Inventive Concept 37. The method according to Inventive Concept 19, further comprising, after applying the dressing to the skin, applying pressure to one or more reservoirs of the dressing that are separate from the fillable insertion chamber, such that a flowable substance contained within the one or more reservoirs flows from the one or more reservoirs to the fillable insertion chamber via one or more passages connecting the one or more reservoirs and the insertion area.

There is still further provided, in accordance with an Inventive Concept 38 of the present invention, a dressing for use with a percutaneous medical device for insertion through skin of a patient at a skin-insertion site, the dressing comprising:
an upper layer, which (a) defines an insertion area having a non-adhesive skin-facing surface configured to be positioned above the skin-insertion site, and (b) is configured to allow passage of the percutaneous medical device through the insertion area;
an adhesive area, which is disposed around at least a portion of a perimeter of the insertion area, and which has a skin-facing surface coated with an adhesive,
   wherein the insertion area is shaped so as to define a fillable insertion chamber between the non-adhesive skin-facing surface and the skin when the dressing is adhered to the skin by at least a portion of the adhesive area;
a flowable substance;
one or more reservoirs, which are separate from the fillable insertion chamber, and contain the flowable substance; and
one or more passages connecting the one or more reservoirs of the flowable substance and the insertion area so as to enable flow of the flowable substance from the one or more reservoirs to the insertion area upon application of pressure to the one or more reservoirs.
Inventive Concept 39. The dressing according to Inventive Concept 38, wherein the insertion area of the upper layer is flexible.
Inventive Concept 40. The dressing according to Inventive Concept 38, wherein the insertion area of the upper layer is elastic.
Inventive Concept 41. The dressing according to Inventive Concept 38, wherein the insertion area of the upper layer is pre-shaped so as to define the fillable insertion chamber.
Inventive Concept 42. The dressing according to Inventive Concept 38, wherein the flowable substance comprises a gel.
Inventive Concept 43. The dressing according to Inventive Concept 38, wherein the flowable substance comprises a cream.
Inventive Concept 44. The dressing according to Inventive Concept 38, wherein the flowable substance comprises a liquid.
Inventive Concept 45. The dressing according to Inventive Concept 38, wherein the flowable substance is configured to change color upon occurrence of one or more of the group of occurrences consisting of: microbial contamination, a change in a pH of the flowable substance, a change in a chemical configuration of the flowable substance, and exposure of the flowable substance to blood.
Inventive Concept 46. The dressing according to Inventive Concept 38, wherein the one or more passages are configured to inhibit the flow of the flowable substance from the one or more reservoirs to the insertion area when one or more respective interiors of the one or more reservoirs are at atmospheric pressure.
Inventive Concept 47. The dressing according to Inventive Concept 46, wherein the one or more passages are configured to prevent the flow of the flowable substance from the one or more reservoirs to the insertion area when the one or more respective interiors of the one or more reservoirs are at atmospheric pressure.
Inventive Concept 48. The dressing according to Inventive Concept 38, wherein the adhesive area entirely surrounds the perimeter of the insertion area except at one or more locations at which the one or more passages connect to the insertion area, and wherein the upper layer is configured to allow passage of the percutaneous medical device through the insertion area by puncturing of the upper layer.
Inventive Concept 49. The dressing according to Inventive Concept 38, wherein the adhesive area is a first adhesive area, and wherein the dressing comprises a second adhesive area that is configured to adhere the one or more reservoirs to the skin.
Inventive Concept 50. The dressing according to Inventive Concept 38, wherein the one or more passages comprise respective one-way valves.
Inventive Concept 51. The dressing according to Inventive Concept 38, wherein an area of the insertion area of the upper layer is at least 0.5 cm2.
Inventive Concept 52. The dressing according to Inventive Concept 38, wherein a volume of the flowable substance is at least 0.25 cc.
Inventive Concept 53. The dressing according to Inventive Concept 52, wherein the volume is at least 3 cc.
Inventive Concept 54. The dressing according to Inventive Concept 38, wherein the insertion area is transparent.
Inventive Concept 55. The dressing according to any one of Inventive Concepts 38-54,
   wherein the insertion area of the upper layer is shaped so as to define a slit having first and second edges,
   wherein an adhesive-area slit-closing portion of the adhesive area is disposed alongside at least the first edge of the slit, and is configured to be adhered to an upper surface of an upper-layer slit-closing portion of the upper layer alongside the second edge of the slit, and
   wherein the upper layer is configured to allow passage of the percutaneous medical device through the insertion area via an opening through the insertion area defined at a junction of respective inner ends of the first and the second edges of the slit, respectively.
Inventive Concept 56. The dressing according to Inventive Concept 55, wherein the dressing is shaped such that adhering of the adhesive-area slit-closing portion of the adhesive area to the upper surface of the upper-layer slit-closing portion of the upper layer forms the fillable insertion chamber.
Inventive Concept 57. The dressing according to Inventive Concept 56, wherein the dressing is shaped such that adhering of the adhesive-area slit-closing portion of the adhesive area to the upper surface of the upper-layer slit-closing portion of the upper layer provides a tented shape to the fillable insertion chamber.
Inventive Concept 58. The dressing according to Inventive Concept 56, wherein the slit is wedge-shaped.
Inventive Concept 59. The dressing according to Inventive Concept 55,
   wherein the dressing further comprises a seal shaped so as to define a seal slit having first and second seal-slit edges, the seal having properties different in at least one respect from those of material of a remainder of the insertion area,
   wherein the seal is coupled to the insertion area at least partially surrounding the opening at the junction, and
   wherein the seal is configured to form a tight seal around the percutaneous medical device when the percutaneous medical device is passed through the seal and the opening through the insertion area, and the adhesive-area slit-closing portion of the adhesive area is adhered to the upper surface of the upper-layer slit-closing portion of the upper layer alongside the second edge of the slit of the insertion area.
Inventive Concept 60. A kit comprising the dressing according to Inventive Concept 55,
   wherein the kit further comprises a sealing support, which is shaped so as to define (a) an upper surface, (b) a lower surface, and (c) a hole, which (i) passes through the sealing support between the upper and the lower surfaces, and (ii) is configured to form a tight seal around the percutaneous medical device when the percutaneous medical device is disposed passing through the hole,
   wherein a sealing-support-adhering portion of the adhesive area is disposed on a skin-facing surface of the insertion area at least partially surrounding the opening at the junction, and
   wherein the sealing-support-adhering portion is configured to adhere to the upper surface of the sealing support when the percutaneous medical device is disposed passing through the hole of the sealing support and the opening through the insertion area defined by the upper layer of the dressing.
Inventive Concept 61. A kit comprising the dressing according to Inventive Concept 55, the kit further comprising the percutaneous medical device,
   wherein the percutaneous medical device comprises a sealing support, which is shaped so as to define an upper surface and a lower surface, wherein the percutaneous medical device is disposed passing through the sealing support such that the sealing support surrounds a portion of the percutaneous medical device,
   wherein a sealing-support-adhering portion of the adhesive area is disposed on a skin-facing surface of the insertion area at least partially surrounding the opening at the junction, and
   wherein the sealing-support-adhering portion is configured to adhere to the upper surface of the sealing support when the percutaneous medical device is disposed passing through the opening through the insertion area defined by the upper layer of the dressing.
Inventive Concept 62. The dressing according to any one of Inventive Concepts 38-54, wherein the flowable substance comprises one or more agents selected from the group of agents consisting of: an antimicrobial agent and an antiseptic agent.
Inventive Concept 63. The dressing according to Inventive Concept 62, wherein the one or more agents comprise an antibacterial.
Inventive Concept 64. The dressing according to any one of Inventive Concepts 38-54, wherein the flowable substance comprises a glue.
Inventive Concept 65. The dressing according to Inventive Concept 64, wherein the flowable substance comprises one or more agents selected from the group of agents consisting of: an antimicrobial agent and an antiseptic agent, and the one or more agents are disposed in the glue.
Inventive Concept 66. The dressing according to any one of Inventive Concepts 38-54, wherein the flowable substance comprises a liquid hardener of an epoxy, and wherein a solid resin of the epoxy coats the non-adhesive skin-facing surface of the insertion area.
Inventive Concept 67. The dressing according to Inventive Concept 66, wherein one or both of the liquid hardener and the solid resin comprise one or more agents selected from the group of agents consisting of: an antimicrobial agent and an antiseptic agent.
Inventive Concept 68. The dressing according to any one of Inventive Concepts 38-54, wherein the flowable substance comprises a liquid resin of an epoxy, and wherein a solid hardener of the epoxy coats the non-adhesive skin-facing surface of the insertion area.
Inventive Concept 69. The dressing according to Inventive Concept 68, wherein one or both of the liquid resin and the solid hardener comprise one or more agents selected from the group of agents consisting of: an antimicrobial agent and an antiseptic agent.
Inventive Concept 70. The dressing according to any one of Inventive Concepts 38-54, wherein the percutaneous medical device is a percutaneous-insertion instrument, and wherein the upper layer is configured to allow passage of the percutaneous-insertion instrument through the insertion area.
Inventive Concept 71. The dressing according to Inventive Concept 70, wherein the percutaneous-insertion instrument is a tube, and wherein the upper layer is configured to allow passage of the tube through the insertion area.
Inventive Concept 72. The dressing according to Inventive Concept 71, wherein the tube is selected from the group of tubes consisting of: a catheter and a cannula, and wherein the upper layer is configured to allow passage of the selected tube through the insertion area.
Inventive Concept 73. The dressing according to Inventive Concept 70, wherein the percutaneous-insertion instrument is selected from the group of instruments consisting of: a cable and a wire, and wherein the upper layer is configured to allow passage of the selected instrument through the insertion area.
Inventive Concept 74. The dressing according to any one of Inventive Concepts 38-54, wherein the percutaneous medical device is an orthopedic percutaneous fixation device, and wherein the upper layer is configured to allow passage of the orthopedic percutaneous fixation device through the insertion area.
Inventive Concept 75. The dressing according to any one of Inventive Concepts 38-54, wherein the adhesive area is disposed around at least 70% of the perimeter of the insertion area.
Inventive Concept 76. The dressing according to Inventive Concept 75, wherein the adhesive area is disposed around at least 90% of the perimeter of the insertion area.
Inventive Concept 77. The dressing according to Inventive Concept 76, wherein the adhesive area entirely surrounds the perimeter of the insertion area, and wherein the upper layer is configured to allow passage of the percutaneous medical device through the insertion area by puncturing of the upper layer.
Inventive Concept 78. The dressing according to any one of Inventive Concepts 38-54, wherein the one or more passages are connected to the insertion area via one or more openings through the insertion area of the upper layer.
Inventive Concept 79. The dressing according to Inventive Concept 78, wherein the adhesive area is disposed around at least 70% of the perimeter of the insertion area.
Inventive Concept 80. The dressing according to Inventive Concept 79, wherein the adhesive area entirely surrounds the perimeter of the insertion area.
Inventive Concept 81. The dressing according to any one of Inventive Concepts 38-54, wherein the upper layer defines, in addition to the insertion area, a peripheral area, which surrounds at least 50% of the perimeter of the insertion area.
Inventive Concept 82. The dressing according to Inventive Concept 81, wherein the peripheral area defines the skin-facing surface coated with the adhesive.
Inventive Concept 83. The dressing according to Inventive Concept 81, further comprising a reservoir-support layer to which the one or more reservoirs are coupled, wherein the dressing defines a predetermined separation border between the peripheral area of the upper layer and the reservoir-support layer.
Inventive Concept 84. The dressing according to Inventive Concept 83, wherein the predetermined separation border is defined by scoring or perforation.
Inventive Concept 85. The dressing according to Inventive Concept 83, wherein the predetermined separation border is defined by an easy tear line.
Inventive Concept 86. The dressing according to Inventive Concept 81, further comprising a peripheral reinforcement, which is coupled to and reinforces the peripheral area of the upper layer.
Inventive Concept 87. The dressing according to Inventive Concept 86, wherein the peripheral reinforcement defines the skin-facing surface coated with the adhesive.
Inventive Concept 88. The dressing according to any one of Inventive Concepts 38-54, further comprising a peripheral reinforcement, which surrounds at least 50% of the perimeter of the insertion area.
Inventive Concept 89. The dressing according to Inventive Concept 88, wherein the peripheral reinforcement defines the skin-facing surface coated with the adhesive.
Inventive Concept 90. The dressing according to any one of Inventive Concepts 38-54, wherein, when the adhesive area is adhered to the skin, the one or more passages are open to the skin such that the skin partially defines the one or more passages.
Inventive Concept 91. The dressing according to Inventive Concept 90, further comprising a removable protective backing that removably covers (a) the non-adhesive skin-facing surface of the insertion area, (b) the adhesive area, and (c) at least a portion of the one or more passages, so as to seal the one or more passages.
Inventive Concept 92. The dressing according to Inventive Concept 91, wherein a portion of an upper surface of the removable protective backing that removably covers the at least a portion of the one or more passages comprises an adhesive that removably adheres to an upper wall of the at least a portion of the one or more passages, so as to seal the one or more passages.
Inventive Concept 93. The dressing according to any one of Inventive Concepts 38-54, wherein the flowable substance has a volume greater than the product of (a) 1 mm and (b) an area of the insertion area of the upper layer.
Inventive Concept 94. The dressing according to Inventive Concept 93, wherein the volume is greater than the product of (a) 3 mm and (b) the area of the insertion area of the upper layer.
Inventive Concept 95. The dressing according to any one of Inventive Concepts 38-54, wherein the dressing is configured such that when the adhesive area is adhered to the skin and the fillable insertion chamber is filled with 60% of the flowable substance from the one or more reservoirs, a greatest height of the flowable substance within the fillable insertion chamber is at least 1 mm.
Inventive Concept 96. The dressing according to Inventive Concept 95, wherein the greatest height is at least 1 mm.
Inventive Concept 97. The dressing according to any one of Inventive Concepts 38-54, wherein the dressing comprises first and second reservoirs, which contain respective first and second portions of the flowable substance.
Inventive Concept 98. The dressing according to Inventive Concept 97, wherein a volume of the first portion of the flowable substance in the first reservoir is greater than the product of (a) 1 mm and (b) an area of the insertion area of the upper layer, and wherein a volume of the second portion of the flowable substance in the second reservoir is greater than the product of (a) 1 mm and (b) the area of the insertion area of the upper layer.
Inventive Concept 99. The dressing according to Inventive Concept 97,
   wherein the flowable substance comprises an epoxy, wherein the first portion of the flowable substance comprises a liquid resin of the epoxy, and wherein the second portion of the flowable substance comprises a liquid hardener of the epoxy, and
   wherein the first reservoir contains the liquid resin, and the second reservoir contains the liquid hardener.
Inventive Concept 100. The dressing according to Inventive Concept 99,
   wherein the flowable substance comprises one or more agents selected from the group of agents consisting of: an antimicrobial agent and an antiseptic agent, and
   wherein the one or more agents are disposed within at least one of the liquid resin and the liquid hardener.
Inventive Concept 101. The dressing according to any one of Inventive Concepts 38-54, wherein the insertion area of the upper layer defines a medical-device penetration region designated for passage of the percutaneous medical device through the insertion area.
Inventive Concept 102. The dressing according to Inventive Concept 101,
   wherein the medical-device penetration region comprises a seal, which has properties different in at least one respect from those of material of a remainder of the insertion area of the upper layer, and
   wherein the seal is configured to form a tight seal around the percutaneous medical device when the percutaneous medical device is passed through the medical-device penetration region.
Inventive Concept 103. The dressing according to any one of Inventive Concepts 38-54, further comprising a removable protective backing that removably covers (a) the non-adhesive skin-facing surface of the insertion area and (b) the adhesive area.
Inventive Concept 104. The dressing according to Inventive Concept 103,
   wherein the adhesive area is a first adhesive area, and wherein the dressing comprises a second adhesive area that is configured to adhere the one or more reservoirs to the skin, and
   wherein the removable protective backing removably covers (a) the non-adhesive skin-facing surface of the insertion area, (b) the first adhesive area, and (c) the second adhesive area.
Inventive Concept 105. A kit comprising the dressing according to any one of Inventive Concepts 38-54, the kit further comprising the percutaneous medical device.
Inventive Concept 106. The kit according to Inventive Concept 105, wherein the percutaneous medical device comprises a percutaneous-insertion instrument.
Inventive Concept 107. The kit according to Inventive Concept 106, wherein the percutaneous-insertion instrument comprises a tube.
Inventive Concept 108. The kit according to Inventive Concept 107, wherein the tube is selected from the group of tubes consisting of: a catheter and a canella.
Inventive Concept 109. The kit according to Inventive Concept 105, wherein the percutaneous-insertion instrument is selected from the group of instruments consisting of: a cable and a wire.
Inventive Concept 110. The kit according to Inventive Concept 105, wherein the percutaneous medical device comprises an orthopedic percutaneous fixation device.

There is additionally provided, in accordance with an Inventive Concept 111 of the present invention, a method comprising:
applying a dressing to skin of a patient by:
   positioning, above a skin-insertion site, a non-adhesive skin-facing surface of an insertion area of an upper layer of the dressing, and
   adhering at least a portion of a skin-facing surface of an adhesive area of the dressing to the skin, such that the insertion area is shaped so as to define a fillable insertion chamber between the non-adhesive skin-facing surface and the skin, wherein the adhesive area is disposed around at least a portion of a perimeter of the insertion area and is coated with an adhesive;
causing a percutaneous medical device to pass through the insertion area;
before or after causing the percutaneous medical device to pass through the insertion area, passing the percutaneous medical device through the skin at the skin-insertion site; and
after applying the dressing to the skin, applying pressure to one or more reservoirs of the dressing that are separate from the fillable insertion chamber, such that a flowable substance contained within the one or more reservoirs flows from the one or more reservoirs to the fillable insertion chamber via one or more passages connecting the one or more reservoirs and the insertion area.
Inventive Concept 112. The method according to Inventive Concept 111, wherein applying the pressure comprises applying the pressure after applying the dressing to the skin and after causing the percutaneous medical device to pass through the insertion area and passing the percutaneous medical device through the skin at the skin-insertion site.
Inventive Concept 113. The method according to Inventive Concept 111, wherein the insertion area of the upper layer is flexible.
Inventive Concept 114. The method according to Inventive Concept 111, wherein the insertion area of the upper layer is elastic.
Inventive Concept 115. The method according to Inventive Concept 111, wherein the insertion area of the upper layer is pre-shaped so as to define the fillable insertion chamber.
Inventive Concept 116. The method according to Inventive Concept 111, wherein causing the percutaneous medical device to pass through the insertion area comprises puncturing the upper layer.
Inventive Concept 117. The method according to Inventive Concept 111,
   wherein the insertion area of the upper layer is shaped so as to define a slit having first and second edges, and an adhesive-area slit-closing portion of the adhesive area is disposed alongside at least the first edge of the slit,
   wherein causing the percutaneous medical device to pass through the insertion area comprises causing the percutaneous medical device to pass through the insertion area via an opening through the insertion area defined at a junction of respective inner ends of the first and the second edges of the slit, respectively, and
   wherein applying the dressing comprises adhering the adhesive-area slit-closing portion of the adhesive area to an upper surface of an upper-layer slit-closing portion of the upper layer alongside the second edge of the slit.
Inventive Concept 118. The method according to Inventive Concept 117, wherein causing the percutaneous medical device to pass through the insertion area via the opening comprises, after passing the percutaneous medical device through the skin at the skin-insertion site, positioning the first and the second edges of the slit around a longitudinal portion of the percutaneous medical device such that the percutaneous medical device passes through the opening.
Inventive Concept 119. The method according to Inventive Concept 117, wherein adhering the adhesive-area slit-closing portion of the adhesive area to the upper surface of the upper-layer slit-closing portion of the upper layer forms the fillable insertion chamber.
Inventive Concept 120. The method according to Inventive Concept 119, wherein adhering the adhesive-area slit-closing portion of the adhesive area to the upper surface of the upper-layer slit-closing portion of the upper layer provides a tented shape to the fillable insertion chamber.
Inventive Concept 121. The method according to Inventive Concept 119, wherein the slit is wedge-shaped.
Inventive Concept 122. The method according to Inventive Concept 117,
   wherein the dressing further includes a seal shaped so as to define a seal slit having first and second seal-slit edges, the seal having properties different in at least one respect from those of material of a remainder of the insertion area,
   wherein the seal is coupled to the insertion area at least partially surrounding the opening at the junction, and
   wherein adhering the adhesive-area slit-closing portion of the adhesive area comprises adhering the adhesive-area slit-closing portion of the adhesive area to the upper surface of the upper-layer slit-closing portion of the upper layer alongside the second edge of the slit such that the seal forms a tight seal around the percutaneous medical device.
Inventive Concept 123. The method according to Inventive Concept 117,
   wherein the method further comprises, before causing the percutaneous medical device to pass through the insertion area, disposing the percutaneous medical device passing through a hole defined by a sealing support such that the hole forms a tight seal around the percutaneous medical device, the hole passing between an upper surface and a lower surface of the sealing support,
   wherein a sealing-support-adhering portion of the adhesive area is disposed on a skin-facing surface of the insertion area at least partially surrounding the opening at the junction, and
   wherein causing the percutaneous medical device to pass through the insertion area via the opening comprises adhering the sealing-support-adhering portion to the upper surface of the sealing support.
Inventive Concept 124. The method according to Inventive Concept 117,
   wherein the percutaneous medical device includes a sealing support, which is shaped so as to define an upper surface and a lower surface, wherein the percutaneous medical device is disposed passing through the sealing support such that the sealing support surrounds a portion of the percutaneous medical device,
   wherein a sealing-support-adhering portion of the adhesive area is disposed on a skin-facing surface of the insertion area at least partially surrounding the opening at the junction, and
   wherein causing the percutaneous medical device to pass through the insertion area via the opening comprises adhering the sealing-support-adhering portion to the upper surface of the sealing support.
Inventive Concept 125. The method according to Inventive Concept 111, wherein the flowable substance includes one or more agents selected from the group of agents consisting of: an antimicrobial agent and an antiseptic agent.
Inventive Concept 126. The method according to Inventive Concept 125, wherein the one or more agents include an antibacterial.
Inventive Concept 127. The method according to Inventive Concept 111, wherein the flowable substance includes a gel.
Inventive Concept 128. The method according to Inventive Concept 111, wherein the flowable substance includes a cream.
Inventive Concept 129. The method according to Inventive Concept 111, wherein the flowable substance includes a liquid.
Inventive Concept 130. The method according to Inventive Concept 111, wherein the flowable substance includes a glue.
Inventive Concept 131. The method according to Inventive Concept 130, wherein the flowable substance includes one or more agents selected from the group of agents consisting of: an antimicrobial agent and an antiseptic agent, and the one or more agents are disposed in the glue.
Inventive Concept 132. The method according to Inventive Concept 111,
   wherein the flowable substance includes a liquid hardener of an epoxy, and
   wherein a solid resin of the epoxy coats the non-adhesive skin-facing surface of the insertion area.
Inventive Concept 133. The method according to Inventive Concept 132, wherein one or both of the liquid hardener and the solid resin include one or more agents selected from the group of agents consisting of: an antimicrobial agent and an antiseptic agent.
Inventive Concept 134. The method according to Inventive Concept 111,
   wherein the flowable substance includes a liquid resin of an epoxy, and
   wherein a solid hardener of the epoxy coats the non-adhesive skin-facing surface of the insertion area.
Inventive Concept 135. The method according to Inventive Concept 134, wherein one or both of the liquid resin and the solid hardener include one or more agents selected from the group of agents consisting of: an antimicrobial agent and an antiseptic agent.
Inventive Concept 136. The method according to Inventive Concept 111, wherein causing the percutaneous medical device to pass through the insertion area comprises causing a percutaneous-insertion instrument to pass through the insertion area.
Inventive Concept 137. The method according to Inventive Concept 136, wherein causing the percutaneous-insertion instrument to pass through the insertion area comprises causing a tube to pass through the insertion area.
Inventive Concept 138. The method according to Inventive Concept 137, wherein the tube is selected from the group of tubes consisting of: a catheter and a cannula, wherein causing the tube to pass through the insertion area comprises causing the selected tube to pass through the insertion area.
Inventive Concept 139. The method according to Inventive Concept 136, wherein the percutaneous-insertion instrument is selected from the group of instruments consisting of: a cable and a wire, and wherein causing the percutaneous-insertion instrument to pass through the insertion area comprises causing selected instrument to pass through the insertion area.
Inventive Concept 140. The method according to Inventive Concept 111, wherein causing the percutaneous medical device to pass through the insertion area comprises causing an orthopedic percutaneous fixation device to pass through the insertion area.
Inventive Concept 141. The method according to Inventive Concept 111, wherein the flowable substance is configured to change color upon occurrence of one or more of the group of occurrences consisting of: microbial contamination, a change in a pH of the flowable substance, a change in a chemical configuration of the flowable substance, and exposure of the flowable substance to blood.
Inventive Concept 142. The method according to Inventive Concept 111, wherein the one or more passages are configured to inhibit the flow of the flowable substance from the one or more reservoirs to the insertion area when one or more respective interiors of the one or more reservoirs are at atmospheric pressure.
Inventive Concept 143. The method according to Inventive Concept 142, wherein the one or more passages are configured to prevent the flow of the flowable substance from the one or more reservoirs to the insertion area when the one or more respective interiors of the one or more reservoirs are at atmospheric pressure.
Inventive Concept 144. The method according to Inventive Concept 111, wherein the adhesive area is disposed around at least 70% of the perimeter of the insertion area.
Inventive Concept 145. The method according to Inventive Concept 144, wherein the adhesive area is disposed around at least 90% of the perimeter of the insertion area.
Inventive Concept 146. The method according to Inventive Concept 145, wherein the adhesive area entirely surrounds the perimeter of the insertion area, and wherein causing the percutaneous medical device to pass through the insertion area comprises puncturing the upper layer.
Inventive Concept 147. The method according to Inventive Concept 111, wherein the adhesive area entirely surrounds the perimeter of the insertion area except at one or more locations at which the one or more passages connect to the insertion area, and wherein causing the percutaneous medical device to pass through the insertion area comprises puncturing the upper layer.
Inventive Concept 148. The method according to Inventive Concept 111, wherein the adhesive area is a first adhesive area, and wherein applying the dressing further comprises adhering the one or more reservoirs to the skin by adhering a second adhesive area of the dressing to the skin.
Inventive Concept 149. The method according to Inventive Concept 111, wherein the one or more passages are connected to the insertion area via one or more openings through the insertion area of the upper layer.
Inventive Concept 150. The method according to Inventive Concept 149, wherein the adhesive area is disposed around at least 70% of the perimeter of the insertion area.
Inventive Concept 151. The method according to Inventive Concept 150, wherein the adhesive area entirely surrounds the perimeter of the insertion area.
Inventive Concept 152. The method according to Inventive Concept 111, wherein the upper layer defines, in addition to the insertion area, a peripheral area, which surrounds at least 50% of the perimeter of the insertion area.
Inventive Concept 153. The method according to Inventive Concept 152, wherein the peripheral area defines the skin-facing surface coated with the adhesive.
Inventive Concept 154. The method according to Inventive Concept 152,
   wherein the dressing further includes a reservoir-support layer to which the one or more reservoirs are coupled,
   wherein the dressing defines a predetermined separation border between the peripheral area of the upper layer and the reservoir-support layer, and
   wherein the method further comprises, after applying the pressure to the one or more reservoirs, removing the one or more reservoirs from the insertion area by separating the reservoir-support layer from the upper layer at the predetermined separation border.
Inventive Concept 155. The method according to Inventive Concept 154, wherein the predetermined separation border is defined by scoring or perforation.
Inventive Concept 156. The method according to Inventive Concept 154, wherein the predetermined separation border is defined by an easy tear line.
Inventive Concept 157. The method according to Inventive Concept 152, wherein the dressing further includes a peripheral reinforcement, which is coupled to and reinforces the peripheral area of the upper layer.
Inventive Concept 158. The method according to Inventive Concept 157, wherein the peripheral reinforcement defines the skin-facing surface coated with the adhesive.
Inventive Concept 159. The method according to Inventive Concept 111, wherein the dressing further includes a peripheral reinforcement, which surrounds at least 50% of the perimeter of the insertion area.
Inventive Concept 160. The method according to Inventive Concept 159, wherein the peripheral reinforcement defines the skin-facing surface coated with the adhesive.
Inventive Concept 161. The method according to Inventive Concept 111, wherein, when the adhesive area is adhered to the skin, the one or more passages are open to the skin such that the skin partially defines the one or more passages.
Inventive Concept 162. The method according to Inventive Concept 161, wherein applying the dressing further comprises, before adhering the at least a portion of the skin-facing surface of the adhesive area to the skin, removing a removable protective backing that removably covers (a) the non-adhesive skin-facing surface of the insertion area, (b) the adhesive area, and (c) at least a portion of the one or more passages, so as to seal the one or more passages.
Inventive Concept 163. The method according to Inventive Concept 162, wherein a portion of an upper surface of the removable protective backing that removably covers the at least a portion of the one or more passages includes an adhesive that removably adheres to an upper wall of the at least a portion of the one or more passages, so as to seal the one or more passages.
Inventive Concept 164. The method according to Inventive Concept 111, wherein the flowable substance has a volume greater than the product of (a) 1 mm and (b) an area of the insertion area of the upper layer.
Inventive Concept 165. The method according to Inventive Concept 164, wherein the volume is greater than the product of (a) 3 mm and (b) the area of the insertion area of the upper layer.
Inventive Concept 166. The method according to Inventive Concept 111, wherein an area of the insertion area of the upper layer is at least 0.5 cm2.
Inventive Concept 167. The method according to Inventive Concept 111, wherein applying the pressure causes the flowable substance to flow to the fillable insertion chamber such that a greatest height of the flowable substance within the fillable insertion chamber is at least 1 mm.
Inventive Concept 168. The method according to Inventive Concept 167, wherein the greatest height is at least 1 mm.
Inventive Concept 169. The method according to Inventive Concept 111, wherein applying the pressure to the one or more reservoirs comprises:
   applying pressure to the one or more reservoirs, such that a first portion of the flowable substance flows from the one or more reservoirs to the fillable insertion chamber; and
   thereafter, after the first portion of the flowable substance has at least partially drained from the fillable insertion chamber, applying pressure to the one or more reservoirs such that a second portion of the flowable substance flows from the one or more reservoirs to the fillable insertion chamber.
Inventive Concept 170. The method according to Inventive Concept 169, further comprising, before applying the pressure to the one or more reservoirs such that the second portion of the flowable substance flows from the one or more reservoirs to the fillable insertion chamber:
   at least partially draining the first portion of the flowable substance from the fillable insertion chamber while the dressing remains at least partially applied to the skin.
Inventive Concept 171. The method according to Inventive Concept 169, wherein the one or more reservoirs are exactly one reservoir, and wherein applying the pressure to the one or more reservoirs comprises:
   applying the pressure to the exactly one reservoir, such that the first portion of the flowable substance flows from the exactly one reservoir to the fillable insertion chamber; and
   thereafter, after the first portion of the flowable substance has at least partially drained from the fillable insertion chamber, applying the pressure to the exactly one reservoir such that a second portion of the flowable substance flows from the exactly one reservoir to the fillable insertion chamber.
Inventive Concept 172. The method according to Inventive Concept 169, wherein a volume of the flowable substance in the one or more reservoirs is greater than the product of (a) 2 and (b) 1 mm and (c) an area of the insertion area of the upper layer.
Inventive Concept 173. The method according to Inventive Concept 111,
   wherein the dressing includes first and second reservoirs, which contain respective first and second portions of the flowable substance, and
   wherein applying the pressure to the one or more reservoirs comprises applying the pressure to the first and the second reservoirs such that the respective first and second portions of the flowable substance flow from the first and the second reservoirs, respectively, to the fillable insertion chamber.
Inventive Concept 174. The method according to Inventive Concept 173, wherein applying the pressure to the first and the second reservoirs comprises:
   applying pressure to the first reservoir, and not to the second reservoir, such that the first portion of the flowable substance flows from the first reservoir to the fillable insertion chamber; and
   thereafter, after the first portion of the flowable substance has at least partially drained from the fillable insertion chamber, applying pressure the second reservoir such that the second portion of the flowable substance flows from the second reservoir to the fillable insertion chamber.
Inventive Concept 175. The method according to Inventive Concept 174, further comprising, before applying the pressure to the second reservoir such that the second portion of the flowable substance flows from the second reservoir to the fillable insertion chamber:
   at least partially draining the first portion of the flowable substance from the fillable insertion chamber while the dressing remains at least partially applied to the skin.
Inventive Concept 176. The method according to Inventive Concept 173, wherein a volume of the first portion of the flowable substance in the first reservoir is greater than the product of (a) 1 mm and (b) an area of the insertion area of the upper layer, and wherein a volume of the second portion of the flowable substance in the second reservoir is greater than the product of (a) 1 mm and (b) the area of the insertion area of the upper layer.
Inventive Concept 177. The method according to Inventive Concept 111,
   wherein the insertion area of the upper layer defines a medical-device penetration region designated for passage of the percutaneous medical device through the insertion area, and
   wherein causing the percutaneous medical device to pass through the insertion area comprises causing the percutaneous medical device to pass through the medical-device penetration region of the insertion area.
Inventive Concept 178. The method according to Inventive Concept 177,
   wherein the medical-device penetration region includes a seal, which has properties different in at least one respect from those of material of a remainder of the insertion area of the upper layer, and
   wherein the seal is configured to form a tight seal around the percutaneous medical device when the percutaneous medical device is passed through the medical-device penetration region.
Inventive Concept 179. The method according to Inventive Concept 111, wherein applying the dressing further comprises, before adhering the at least a portion of the skin-facing surface of the adhesive area to the skin, removing a removable protective backing that removably covers (a) the non-adhesive skin-facing surface of the insertion area and (b) the adhesive area.
Inventive Concept 180. The method according to Inventive Concept 179,
   wherein the adhesive area is a first adhesive area, and wherein the dressing includes a second adhesive area that is configured to adhere the one or more reservoirs to the skin, and
   wherein the removable protective backing removably covers (a) the non-adhesive skin-facing surface of the insertion area, (b) the first adhesive area, and (c) the second adhesive area.
Inventive Concept 181. The method according to Inventive Concept 111, wherein the insertion area is transparent.

There is yet additionally provided, in accordance with an Inventive Concept 182 of the present invention, a dressing for covering an area of skin of a patient, the dressing comprising:
an upper layer, which defines a central area having a non-adhesive skin-facing surface configured to be positioned above the skin area;
an adhesive area, which is disposed around at least a portion of a perimeter of the central area, and which has a skin-facing surface coated with an adhesive,
   wherein the central area is shaped so as to define a fillable chamber between the non-adhesive skin-facing surface and the skin when the dressing is adhered to the skin by at least a portion of the adhesive area;
a flowable substance;
one or more reservoirs, which are separate from the fillable chamber, and contain the flowable substance; and
one or more passages connecting the one or more reservoirs of the flowable substance and the central area so as to enable flow of the flowable substance from the one or more reservoirs to the central area upon application of pressure to the one or more reservoirs.
Inventive Concept 183. The dressing according to Inventive Concept 182, wherein the central area of the upper layer is flexible.
Inventive Concept 184. The dressing according to Inventive Concept 182, wherein the central area of the upper layer is elastic.
Inventive Concept 185. The dressing according to Inventive Concept 182, wherein the central area of the upper layer is pre-shaped so as to define the fillable chamber.
Inventive Concept 186. The dressing according to Inventive Concept 182, wherein the flowable substance comprises a gel.
Inventive Concept 187. The dressing according to Inventive Concept 182, wherein the flowable substance comprises a cream.
Inventive Concept 188. The dressing according to Inventive Concept 182, wherein the flowable substance comprises a liquid.
Inventive Concept 189. The dressing according to Inventive Concept 182, wherein the flowable substance is configured to change color upon occurrence of one or more of the group of occurrences consisting of: microbial contamination, a change in a pH of the flowable substance, a change in a chemical configuration of the flowable substance, and exposure of the flowable substance to blood.
Inventive Concept 190. The dressing according to Inventive Concept 182, wherein the one or more passages are configured to inhibit the flow of the flowable substance from the one or more reservoirs to the central area when one or more respective interiors of the one or more reservoirs are at atmospheric pressure.
Inventive Concept 191. The dressing according to Inventive Concept 190, wherein the one or more passages are configured to prevent the flow of the flowable substance from the one or more reservoirs to the central area when the one or more respective interiors of the one or more reservoirs are at atmospheric pressure.
Inventive Concept 192. The dressing according to Inventive Concept 182, wherein the adhesive area is a first adhesive area, and wherein the dressing comprises a second adhesive area that is configured to adhere the one or more reservoirs to the skin.
Inventive Concept 193. The dressing according to Inventive Concept 182, wherein the one or more passages comprise respective one-way valves.
Inventive Concept 194. The dressing according to Inventive Concept 182, wherein an area of the central area of the upper layer is at least 0.5 cm2.
Inventive Concept 195. The dressing according to Inventive Concept 182, wherein a volume of the flowable substance is at least 0.25 cc.
Inventive Concept 196. The dressing according to Inventive Concept 195, wherein the volume is at least 3 cc.
Inventive Concept 197. The dressing according to Inventive Concept 182, wherein the central area is transparent.
Inventive Concept 198. The dressing according to any one of Inventive Concepts 182-197, wherein the flowable substance comprises one or more agents selected from the group of agents consisting of: an antimicrobial agent and an antiseptic agent.
Inventive Concept 199. The dressing according to Inventive Concept 198, wherein the one or more agents comprise an antibacterial.
Inventive Concept 200. The dressing according to any one of Inventive Concepts 182-197, wherein the adhesive area is disposed around at least 70% of the perimeter of the central area.
Inventive Concept 201. The dressing according to Inventive Concept 200, wherein the adhesive area is disposed around at least 90% of the perimeter of the central area.
Inventive Concept 202. The dressing according to Inventive Concept 201, wherein the adhesive area entirely surrounds the perimeter of the central area.
Inventive Concept 203. The dressing according to any one of Inventive Concepts 182-197, wherein the one or more passages are connected to the central area via one or more openings through the central area of the upper layer.
Inventive Concept 204. The dressing according to Inventive Concept 203, wherein the adhesive area is disposed around at least 70% of the perimeter of the central area.
Inventive Concept 205. The dressing according to Inventive Concept 204, wherein the adhesive area entirely surrounds the perimeter of the central area.
Inventive Concept 206. The dressing according to any one of Inventive Concepts 182-197, wherein the upper layer defines, in addition to the central area, a peripheral area, which surrounds at least 50% of the perimeter of the central area.
Inventive Concept 207. The dressing according to Inventive Concept 206, wherein the peripheral area defines the skin-facing surface coated with the adhesive.
Inventive Concept 208. The dressing according to Inventive Concept 206, further comprising a peripheral reinforcement, which is coupled to and reinforces the peripheral area of the upper layer.
Inventive Concept 209. The dressing according to Inventive Concept 208, wherein the peripheral reinforcement defines the skin-facing surface coated with the adhesive.
Inventive Concept 210. The dressing according to Inventive Concept 206, further comprising a reservoir-support layer to which the one or more reservoirs are coupled, wherein the dressing defines a predetermined separation border between the peripheral area of the upper layer and the reservoir-support layer.
Inventive Concept 211. The dressing according to Inventive Concept 210, wherein the predetermined separation border is defined by scoring or perforation.
Inventive Concept 212. The dressing according to Inventive Concept 210, wherein the predetermined separation border is defined by an easy tear line.
Inventive Concept 213. The dressing according to any one of Inventive Concepts 182-197, further comprising a peripheral reinforcement, which surrounds at least 50% of the perimeter of the central area.
Inventive Concept 214. The dressing according to Inventive Concept 213, wherein the peripheral reinforcement defines the skin-facing surface coated with the adhesive.
Inventive Concept 215. The dressing according to any one of Inventive Concepts 182-197, wherein, when the adhesive area is adhered to the skin, the one or more passages are open to the skin such that the skin partially defines the one or more passages.
Inventive Concept 216. The dressing according to Inventive Concept 215, further comprising a removable protective backing that removably covers (a) the non-adhesive skin-facing surface of the central area, (b) the adhesive area, and (c) at least a portion of the one or more passages, so as to seal the one or more passages.
Inventive Concept 217. The dressing according to Inventive Concept 216, wherein a portion of an upper surface of the removable protective backing that removably covers the at least a portion of the one or more passages comprises an adhesive that removably adheres to an upper wall of the at least a portion of the one or more passages, so as to seal the one or more passages.
Inventive Concept 218. The dressing according to any one of Inventive Concepts 182-197, wherein the flowable substance has a volume greater than the product of (a) 1 mm and (b) an area of the central area of the upper layer.
Inventive Concept 219. The dressing according to Inventive Concept 218, wherein the volume is greater than the product of (a) 3 mm and (b) the area of the central area of the upper layer.
Inventive Concept 220. The dressing according to any one of Inventive Concepts 182-197, wherein the dressing is configured such that when the adhesive area is adhered to the skin and the fillable chamber is filled with 60% of the flowable substance from the one or more reservoirs, a greatest height of the flowable substance within the fillable chamber is at least 1 mm.
Inventive Concept 221. The dressing according to Inventive Concept 220, wherein the greatest height is at least 1 mm.
Inventive Concept 222. The dressing according to any one of Inventive Concepts 182-197, wherein the dressing comprises first and second reservoirs, which contain respective first and second portions of the flowable substance.
Inventive Concept 223. The dressing according to Inventive Concept 222, wherein a volume of the first portion of the flowable substance in the first reservoir is greater than the product of (a) 1 mm and (b) an area of the central area of the upper layer, and wherein a volume of the second portion of the flowable substance in the second reservoir is greater than the product of (a) 1 mm and (b) the area of the central area of the upper layer.
Inventive Concept 224. The dressing according to any one of Inventive Concepts 182-197, further comprising a removable protective backing that removably covers (a) the non-adhesive skin-facing surface of the central area and (b) the adhesive area.
Inventive Concept 225. The dressing according to Inventive Concept 224,
   wherein the adhesive area is a first adhesive area, and wherein the dressing comprises a second adhesive area that is configured to adhere the one or more reservoirs to the skin, and
   wherein the removable protective backing removably covers (a) the non-adhesive skin-facing surface of the central area, (b) the first adhesive area, and (c) the second adhesive area.
Inventive Concept 226. The dressing according to any one of Inventive Concepts 182-225, for use with a percutaneous medical device for insertion through the skin area at a skin-insertion site,
   wherein the central area comprises an insertion area having the non-adhesive skin-facing surface configured to be positioned above the skin-insertion site, and
   wherein the upper layer is configured to allow passage of the percutaneous medical device through the insertion area.
Inventive Concept 227. The dressing according to Inventive Concept 226, wherein the adhesive area entirely surrounds the perimeter of the central area except at one or more locations at which the one or more passages connect to the central area, and wherein the upper layer is configured to allow passage of the percutaneous medical device through the insertion area by puncturing of the upper layer.
Inventive Concept 228. The dressing according to Inventive Concept 226,
   wherein the insertion area of the upper layer is shaped so as to define a slit having first and second edges,
   wherein an adhesive-area slit-closing portion of the adhesive area is disposed alongside at least the first edge of the slit, and is configured to be adhered to an upper surface of an upper-layer slit-closing portion of the upper layer alongside the second edge of the slit, and
   wherein the upper layer is configured to allow passage of the percutaneous medical device through the insertion area via an opening through the insertion area defined at a junction of respective inner ends of the first and the second edges of the slit, respectively.
Inventive Concept 229. The dressing according to Inventive Concept 228, wherein the dressing is shaped such that adhering of the adhesive-area slit-closing portion of the adhesive area to the upper surface of the upper-layer slit-closing portion of the upper layer forms the fillable chamber.
Inventive Concept 230. The dressing according to Inventive Concept 229, wherein the dressing is shaped such that adhering of the adhesive-area slit-closing portion of the adhesive area to the upper surface of the upper-layer slit-closing portion of the upper layer provides a tented shape to the fillable chamber.
Inventive Concept 231. The dressing according to Inventive Concept 229, wherein the slit is wedge-shaped.
Inventive Concept 232. The dressing according to Inventive Concept 226, wherein the flowable substance comprises a glue.
Inventive Concept 233. The dressing according to Inventive Concept 232, wherein the flowable substance comprises one or more agents selected from the group of agents consisting of: an antimicrobial agent and an antiseptic agent, and the one or more agents are disposed in the glue.
Inventive Concept 234. The dressing according to Inventive Concept 226,
   wherein the flowable substance comprises a liquid hardener of an epoxy, and
   wherein a solid resin of the epoxy coats the non-adhesive skin-facing surface of the central area.
Inventive Concept 235. The dressing according to Inventive Concept 234, wherein one or both of the liquid hardener and the solid resin comprise one or more agents selected from the group of agents consisting of: an antimicrobial agent and an antiseptic agent.
Inventive Concept 236. The dressing according to Inventive Concept 226,
   wherein the flowable substance comprises a liquid resin of an epoxy, and
   wherein a solid hardener of the epoxy coats the non-adhesive skin-facing surface of the central area.
Inventive Concept 237. The dressing according to Inventive Concept 236, wherein one or both of the liquid resin and the solid hardener comprise one or more agents selected from the group of agents consisting of: an antimicrobial agent and an antiseptic agent.
Inventive Concept 238. The dressing according to Inventive Concept 226, wherein the percutaneous medical device is a percutaneous-insertion instrument, and wherein the upper layer is configured to allow passage of the percutaneous-insertion instrument through the insertion area.
Inventive Concept 239. The dressing according to Inventive Concept 238, wherein the percutaneous-insertion instrument is a tube, and wherein the upper layer is configured to allow passage of the tube through the insertion area.
Inventive Concept 240. The dressing according to Inventive Concept 239, wherein the tube is selected from the group of tubes consisting of: a catheter and a cannula, and wherein the upper layer is configured to allow passage of the selected tube through the insertion area.
Inventive Concept 241. The dressing according to Inventive Concept 238, wherein the percutaneous-insertion instrument is selected from the group of instruments consisting of: a cable and a wire, and wherein the upper layer is configured to allow passage of the selected instrument through the insertion area.
Inventive Concept 242. The dressing according to Inventive Concept 226, wherein the percutaneous medical device is an orthopedic percutaneous fixation device, and wherein the upper layer is configured to allow passage of the orthopedic percutaneous fixation device through the insertion area.
Inventive Concept 243. The dressing according to Inventive Concept 226, wherein the adhesive area entirely surrounds the perimeter of the central area, and wherein the upper layer is configured to allow passage of the percutaneous medical device through the insertion area by puncturing of the upper layer.
Inventive Concept 244. The dressing according to Inventive Concept 226,
   wherein the dressing comprises first and second reservoirs, which contain respective first and second portions of the flowable substance,
   wherein the flowable substance comprises an epoxy, wherein the first portion of the flowable substance comprises a liquid resin of the epoxy, and wherein the second portion of the flowable substance comprises a liquid hardener of the epoxy, and
   wherein the first reservoir contains the liquid resin, and the second reservoir contains the liquid hardener.
Inventive Concept 245. The dressing according to Inventive Concept 244,
   wherein the flowable substance comprises one or more agents selected from the group of agents consisting of: an antimicrobial agent and an antiseptic agent, and
   wherein the one or more agents are disposed within at least one of the liquid resin and the liquid hardener.
Inventive Concept 246. A kit comprising the dressing according to Inventive Concept 226, the kit further comprising the percutaneous medical device.
Inventive Concept 247. The kit according to Inventive Concept 246, wherein the percutaneous medical device comprises a percutaneous-insertion instrument.
Inventive Concept 248. The kit according to Inventive Concept 247, wherein the percutaneous-insertion instrument comprises a tube.
Inventive Concept 249. The kit according to Inventive Concept 248, wherein the tube is selected from the group of tubes consisting of: a catheter and a canella.
Inventive Concept 250. The kit according to Inventive Concept 246, wherein the percutaneous-insertion instrument is selected from the group of instruments consisting of: a cable and a wire.
Inventive Concept 251. The kit according to Inventive Concept 246, wherein the percutaneous medical device comprises an orthopedic percutaneous fixation device.
Inventive Concept 252. The dressing according to any one of Inventive Concepts 182-225, for covering a wound of the skin area, wherein the central area comprises a wound-covering area having the non-adhesive skin-facing surface configured to be positioned above the wound.
Inventive Concept 253. The dressing according to Inventive Concept 252, wherein the dressing is shaped as a finger cot.

There is also provided, in accordance with an Inventive Concept 254 of the present invention, a method comprising:
applying a dressing to skin of a patient by:
   positioning, above an area of the skin, a non-adhesive skin-facing surface of a central area of an upper layer of the dressing, and
   adhering at least a portion of a skin-facing surface of an adhesive area of the dressing to the skin, such that the central area is shaped so as to define a fillable chamber between the non-adhesive skin-facing surface and the skin, wherein the adhesive area is disposed around at least a portion of a perimeter of the central area and is coated with an adhesive; and
after applying the dressing to the skin, applying pressure to one or more reservoirs of the dressing that are separate from the fillable chamber, such that a flowable substance contained within the one or more reservoirs flows from the one or more reservoirs to the fillable chamber via one or more passages connecting the one or more reservoirs and the central area.
Inventive Concept 255. The method according to Inventive Concept 254, wherein the central area of the upper layer is flexible.
Inventive Concept 256. The method according to Inventive Concept 254, wherein the central area of the upper layer is elastic.
Inventive Concept 257. The method according to Inventive Concept 254, wherein the central area of the upper layer is pre-shaped so as to define the fillable chamber.
Inventive Concept 258. The method according to Inventive Concept 254, wherein the flowable substance includes one or more agents selected from the group of agents consisting of: an antimicrobial agent and an antiseptic agent.
Inventive Concept 259. The method according to Inventive Concept 258, wherein the one or more agents include an antibacterial.
Inventive Concept 260. The method according to Inventive Concept 254, wherein the flowable substance includes a gel.
Inventive Concept 261. The method according to Inventive Concept 254, wherein the flowable substance includes a cream.
Inventive Concept 262. The method according to Inventive Concept 254, wherein the flowable substance includes a liquid.
Inventive Concept 263. The method according to Inventive Concept 254, wherein the flowable substance includes a glue.
Inventive Concept 264. The method according to Inventive Concept 263, wherein the flowable substance includes one or more agents selected from the group of agents consisting of: an antimicrobial agent and an antiseptic agent, and the one or more agents are disposed in the glue.
Inventive Concept 265. The method according to Inventive Concept 254, wherein the flowable substance is configured to change color upon occurrence of one or more of the group of occurrences consisting of: microbial contamination, a change in a pH of the flowable substance, a change in a chemical configuration of the flowable substance, and exposure of the flowable substance to blood.
Inventive Concept 266. The method according to Inventive Concept 254, wherein the one or more passages are configured to inhibit the flow of the flowable substance from the one or more reservoirs to the central area when one or more respective interiors of the one or more reservoirs are at atmospheric pressure.
Inventive Concept 267. The method according to Inventive Concept 266, wherein the one or more passages are configured to prevent the flow of the flowable substance from the one or more reservoirs to the central area when the one or more respective interiors of the one or more reservoirs are at atmospheric pressure.
Inventive Concept 268. The method according to Inventive Concept 254, wherein the adhesive area is disposed around at least 70% of the perimeter of the central area.
Inventive Concept 269. The method according to Inventive Concept 268, wherein the adhesive area is disposed around at least 90% of the perimeter of the central area.
Inventive Concept 270. The method according to Inventive Concept 269, wherein the adhesive area entirely surrounds the perimeter of the central area.
Inventive Concept 271. The method according to Inventive Concept 254, wherein the adhesive area is a first adhesive area, and wherein applying the dressing further comprises adhering the one or more reservoirs to the skin by adhering a second adhesive area of the dressing to the skin.
Inventive Concept 272. The method according to Inventive Concept 254, wherein the one or more passages are connected to the central area via one or more openings through the central area of the upper layer.
Inventive Concept 273. The method according to Inventive Concept 272, wherein the adhesive area is disposed around at least 70% of the perimeter of the central area.
Inventive Concept 274. The method according to Inventive Concept 273, wherein the adhesive area entirely surrounds the perimeter of the central area.
Inventive Concept 275. The method according to Inventive Concept 254, wherein the upper layer defines, in addition to the central area, a peripheral area, which surrounds at least 50% of the perimeter of the central area.
Inventive Concept 276. The method according to Inventive Concept 275, wherein the peripheral area defines the skin-facing surface coated with the adhesive.
Inventive Concept 277. The method according to Inventive Concept 275, wherein the dressing further includes a peripheral reinforcement, which is coupled to and reinforces the peripheral area of the upper layer.
Inventive Concept 278. The method according to Inventive Concept 277, wherein the peripheral reinforcement defines the skin-facing surface coated with the adhesive.
Inventive Concept 279. The method according to Inventive Concept 275,
   wherein the dressing further includes a reservoir-support layer to which the one or more reservoirs are coupled,
   wherein the dressing defines a predetermined separation border between the peripheral area of the upper layer and the reservoir-support layer, and
   wherein the method further comprises, after applying the pressure to the one or more reservoirs, removing the one or more reservoirs from the central area by separating the reservoir-support layer from the upper layer at the predetermined separation border.
Inventive Concept 280. The method according to Inventive Concept 279, wherein the predetermined separation border is defined by scoring or perforation.
Inventive Concept 281. The method according to Inventive Concept 279, wherein the predetermined separation border is defined by an easy tear line.
Inventive Concept 282. The method according to Inventive Concept 254, wherein the dressing further includes a peripheral reinforcement, which surrounds at least 50% of the perimeter of the central area.
Inventive Concept 283. The method according to Inventive Concept 282, wherein the peripheral reinforcement defines the skin-facing surface coated with the adhesive.
Inventive Concept 284. The method according to Inventive Concept 254, wherein, when the adhesive area is adhered to the skin, the one or more passages are open to the skin such that the skin partially defines the one or more passages.
Inventive Concept 285. The method according to Inventive Concept 284, wherein applying the dressing further comprises, before adhering the at least a portion of the skin-facing surface of the adhesive area to the skin, removing a removable protective backing that removably covers (a) the non-adhesive skin-facing surface of the central area, (b) the adhesive area, and (c) at least a portion of the one or more passages, so as to seal the one or more passages.
Inventive Concept 286. The method according to Inventive Concept 285, wherein a portion of an upper surface of the removable protective backing that removably covers the at least a portion of the one or more passages includes an adhesive that removably adheres to an upper wall of the at least a portion of the one or more passages, so as to seal the one or more passages.
Inventive Concept 287. The method according to Inventive Concept 254, wherein the flowable substance has a volume greater than the product of (a) 1 mm and (b) an area of the central area of the upper layer.
Inventive Concept 288. The method according to Inventive Concept 287, wherein the volume is greater than the product of (a) 3 mm and (b) the area of the central area of the upper layer.
Inventive Concept 289. The method according to Inventive Concept 254, wherein an area of the central area of the upper layer is at least 0.5 cm2.
Inventive Concept 290. The method according to Inventive Concept 254, wherein applying the pressure causes the flowable substance to flow to the fillable chamber such that a greatest height of the flowable substance within the fillable chamber is at least 1 mm.
Inventive Concept 291. The method according to Inventive Concept 290, wherein the greatest height is at least 1 mm.
Inventive Concept 292. The method according to Inventive Concept 254, wherein applying the pressure to the one or more reservoirs comprises:
   applying pressure to the one or more reservoirs, such that a first portion of the flowable substance flows from the one or more reservoirs to the fillable insertion chamber; and
   thereafter, after the first portion of the flowable substance has at least partially drained from the fillable insertion chamber, applying pressure the one or more reservoirs such that a second portion of the flowable substance flows from the one or more reservoirs to the fillable insertion chamber.
Inventive Concept 293. The method according to Inventive Concept 292, further comprising, before applying the pressure to the one or more reservoirs such that the second portion of the flowable substance flows from the one or more reservoirs to the fillable insertion chamber:
   at least partially draining the first portion of the flowable substance from the fillable insertion chamber while the dressing remains at least partially applied to the skin.
Inventive Concept 294. The method according to Inventive Concept 292, wherein the one or more reservoirs are exactly one reservoir, and wherein applying the pressure to the one or more reservoirs comprises:
   applying the pressure to the exactly one reservoir, such that the first portion of the flowable substance flows from the exactly one reservoir to the fillable insertion chamber; and
   thereafter, after the first portion of the flowable substance has at least partially drained from the fillable insertion chamber, applying the pressure to the exactly one reservoir such that a second portion of the flowable substance flows from the exactly one reservoir to the fillable insertion chamber.
Inventive Concept 295. The method according to Inventive Concept 292, wherein a volume of the flowable substance in the one or more reservoirs is greater than the product of (a) 2 and (b) 1 mm and (c) an area of the insertion area of the upper layer.
Inventive Concept 296. The method according to Inventive Concept 254,
   wherein the dressing includes first and second reservoirs, which contain respective first and second portions of the flowable substance, and
   wherein applying the pressure to the one or more reservoirs comprises applying the pressure to the first and the second reservoirs such that the respective first and second portions of the flowable substance flow from the first and the second reservoirs, respectively, to the fillable chamber.
Inventive Concept 297. The method according to Inventive Concept 296, wherein applying the pressure to the first and the second reservoirs comprises:
   applying pressure to the first reservoir, and not to the second reservoir, such that the first portion of the flowable substance flows from the first reservoir to the fillable chamber; and
   thereafter, after the first portion of the flowable substance has at least partially drained from the fillable insertion chamber, applying pressure the second reservoir such that the second portion of the flowable substance flows from the second reservoir to the fillable chamber.
Inventive Concept 298. The method according to Inventive Concept 297, further comprising, before applying the pressure to the second reservoir such that the second portion of the flowable substance flows from the second reservoir to the fillable insertion chamber:
   at least partially draining the first portion of the flowable substance from the fillable insertion chamber while the dressing remains at least partially applied to the skin.
Inventive Concept 299. The method according to Inventive Concept 296, wherein a volume of the first portion of the flowable substance in the first reservoir is greater than the product of (a) 1 mm and (b) an area of the central area of the upper layer, and wherein a volume of the second portion of the flowable substance in the second reservoir is greater than the product of (a) 1 mm and (b) the area of the central area of the upper layer.
Inventive Concept 300. The method according to Inventive Concept 254, wherein applying the dressing further comprises, before adhering the at least a portion of the skin-facing surface of the adhesive area to the skin, removing a removable protective backing that removably covers (a) the non-adhesive skin-facing surface of the central area and (b) the adhesive area.
Inventive Concept 301. The method according to Inventive Concept 300,
   wherein the adhesive area is a first adhesive area, and wherein the dressing includes a second adhesive area that is configured to adhere the one or more reservoirs to the skin, and
   wherein the removable protective backing removably covers (a) the non-adhesive skin-facing surface of the central area, (b) the first adhesive area, and (c) the second adhesive area.
Inventive Concept 302. The method according to Inventive Concept 254, wherein the central area is transparent.
Inventive Concept 303. The method according to Inventive Concept 254,
   wherein the central area comprises an insertion area having the non-adhesive skin-facing surface,
   wherein positioning the non-adhesive skin-facing surface of the central area above the skin area comprises positioning the non-adhesive skin-facing surface of the central area above a skin-insertion site of the skin area, and
   wherein the method further comprises:
      causing a percutaneous medical device to pass through the insertion area; and
      before or after causing the percutaneous medical device to pass through the insertion area, passing the percutaneous medical device through the skin at the skin-insertion site.
Inventive Concept 304. The method according to Inventive Concept 303, wherein applying the pressure comprises applying the pressure after applying the dressing to the skin and after causing the percutaneous medical device to pass through the insertion area and passing the percutaneous medical device through the skin at the skin-insertion site.
Inventive Concept 305. The method according to Inventive Concept 303, wherein causing the percutaneous medical device to pass through the insertion area comprises puncturing the upper layer.
Inventive Concept 306. The method according to Inventive Concept 303,
   wherein the insertion area of the upper layer is shaped so as to define a slit having first and second edges, and an adhesive-area slit-closing portion of the adhesive area is disposed alongside at least the first edge of the slit,
   wherein causing the percutaneous medical device to pass through the insertion area comprises causing the percutaneous medical device to pass through the insertion area via an opening through the insertion area defined at a junction of respective inner ends of the first and the second edges of the slit, respectively, and
   wherein applying the dressing comprises adhering the adhesive-area slit-closing portion of the adhesive area to an upper surface of an upper-layer slit-closing portion of the upper layer alongside the second edge of the slit.
Inventive Concept 307. The method according to Inventive Concept 306, wherein causing the percutaneous medical device to pass through the insertion area via the opening comprises, after passing the percutaneous medical device through the skin at the skin-insertion site, positioning the first and the second edges of the slit around a longitudinal portion of the percutaneous medical device such that the percutaneous medical device passes through the opening.
Inventive Concept 308. The method according to Inventive Concept 306, wherein adhering the adhesive-area slit-closing portion of the adhesive area to the upper surface of the upper-layer slit-closing portion of the upper layer forms the fillable chamber.
Inventive Concept 309. The method according to Inventive Concept 308, wherein adhering the adhesive-area slit-closing portion of the adhesive area to the upper surface of the upper-layer slit-closing portion of the upper layer provides a tented shape to the fillable chamber.
Inventive Concept 310. The method according to Inventive Concept 308, wherein the slit is wedge-shaped.
Inventive Concept 311. The method according to Inventive Concept 303, wherein the flowable substance includes a liquid hardener of an epoxy, and wherein a solid resin of the epoxy coats the non-adhesive skin-facing surface of the insertion area.
Inventive Concept 312. The method according to Inventive Concept 311, wherein one or both of the liquid hardener and the solid resin include one or more agents selected from the group of agents consisting of: an antimicrobial agent and an antiseptic agent.
Inventive Concept 313. The method according to Inventive Concept 303,
   wherein the flowable substance includes a liquid resin of an epoxy, and
   wherein a solid hardener of the epoxy coats the non-adhesive skin-facing surface of the insertion area.
Inventive Concept 314. The method according to Inventive Concept 313, wherein one or both of the liquid resin and the solid hardener include one or more agents selected from the group of agents consisting of: an antimicrobial agent and an antiseptic agent.
Inventive Concept 315. The method according to Inventive Concept 303, wherein causing the percutaneous medical device to pass through the insertion area comprises causing a percutaneous-insertion instrument to pass through the insertion area.
Inventive Concept 316. The method according to Inventive Concept 315, wherein causing the percutaneous-insertion instrument to pass through the insertion area comprises causing a tube to pass through the insertion area.
Inventive Concept 317. The method according to Inventive Concept 316, wherein the tube is selected from the group of tubes consisting of: a catheter and a cannula, wherein causing the tube to pass through the insertion area comprises causing the selected tube to pass through the insertion area.
Inventive Concept 318. The method according to Inventive Concept 315, wherein the percutaneous-insertion instrument is selected from the group of instruments consisting of: a cable and a wire, and wherein causing the percutaneous-insertion instrument to pass through the insertion area comprises causing selected instrument to pass through the insertion area.
Inventive Concept 319. The method according to Inventive Concept 303, wherein causing the percutaneous medical device to pass through the insertion area comprises causing an orthopedic percutaneous fixation device to pass through the insertion area.
Inventive Concept 320. The method according to Inventive Concept 303, wherein the adhesive area entirely surrounds the perimeter of the central area, and wherein causing the percutaneous medical device to pass through the insertion area comprises puncturing the upper layer.
Inventive Concept 321. The method according to Inventive Concept 303, wherein the adhesive area entirely surrounds the perimeter of the central area except at one or more locations at which the one or more passages connect to the central area, and wherein causing the percutaneous medical device to pass through the insertion area comprises puncturing the upper layer.
Inventive Concept 322. The method according to Inventive Concept 254, wherein the central area includes a wound-covering area, and wherein positioning the non-adhesive skin-facing surface of the central area above the skin area comprises positioning the non-adhesive skin-facing surface of the wound-covering area over a wound of the skin area.
Inventive Concept 323. The method according to Inventive Concept 322, wherein the wound is a wound of a finger of the patient, wherein the dressing is shaped as a finger cot, and wherein positioning the non-adhesive skin-facing surface of the central area over the wound comprises placing the finger cot on the finger.

The present invention will be more fully understood from the following detailed description of embodiments thereof, taken together with the drawings, in which:

### BRIEF DESCRIPTION OF THE DRAWINGS

FIGS. 1A and 1B are schematic isometric and cross-sectional views of a dressing, in accordance with an application of the present invention;

FIGS. 2A-E are schematic illustrations of a method using the dressing of FIGS. 1A-B, in accordance with an application of the present invention;

FIGS. 3A and 3B are schematic isometric views of two configurations of another dressing, in accordance with respective applications of the present invention;

FIGS. 4A-D are schematic isometric views of yet another dressing, in accordance with an application of the present invention;

FIGS. 5A-B are schematic illustrations of still another dressing, in accordance with an application of the present invention;

FIGS. 6A and 6B are schematic illustrations of respective kits, in accordance with respective applications of the present invention;

FIG. 7 is a schematic illustration of the dressing of the kit of FIG. 6A after application to the skin, in accordance with an application of the present invention;

FIG. 8 is schematic illustration of still another dressing, in accordance with an application of the present invention;

FIG. 9 is schematic illustration of another dressing, in accordance with an application of the present invention;

FIG. 10 is a schematic isometric view of still another dressing, in accordance with an application of the present invention;

FIG. 11 is a schematic isometric view of a dressing for covering a wound of an area of skin of a patient, in accordance with an application of the present invention;

FIG. 12A is a schematic cross-sectional view of another dressing, in accordance with an application of the present invention;

FIG. 12B is a schematic illustration of a method for applying the dressing of FIG. 12A to the skin, in accordance with an application of the present invention;

FIG. 13 is a schematic cross-sectional view of still another dressing, in accordance with an application of the present invention;

FIGS. 14A-B are schematic isometric and cross-sectional views of another dressing for covering a wound of an area of skin of a finger of a patient, in accordance with an application of the present invention; and

FIGS. 15A-B are schematic illustrations of the dressing of FIGS. 14A-B applied to the finger of the patient, in accordance with an application of the present invention.

### DETAILED DESCRIPTION OF APPLICATIONS

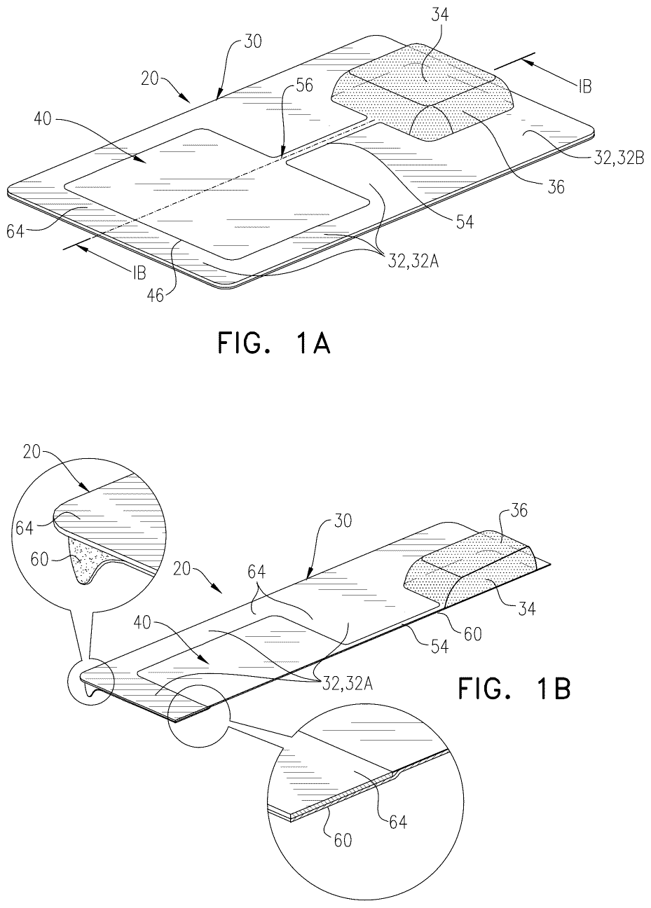

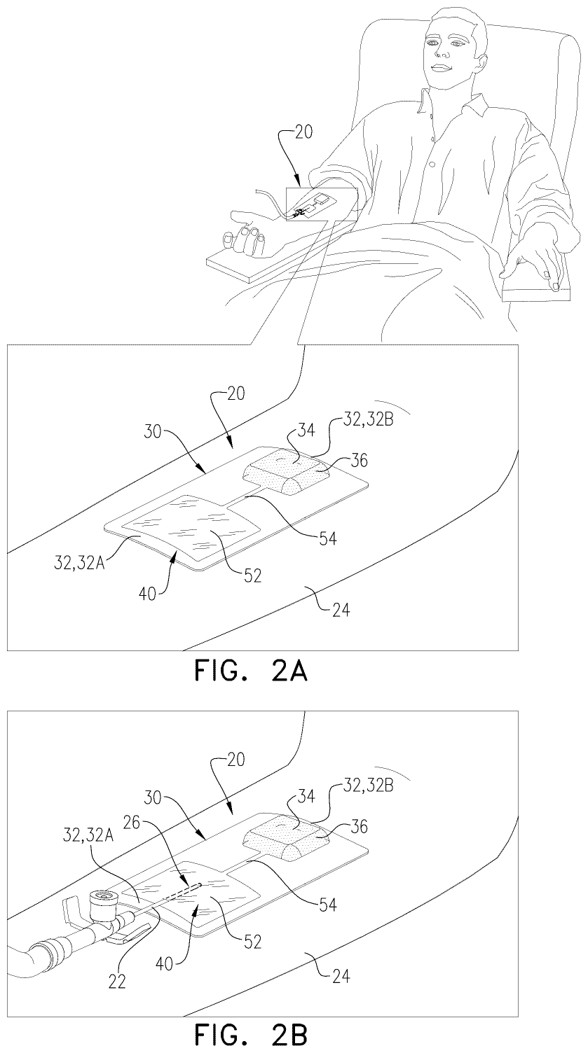

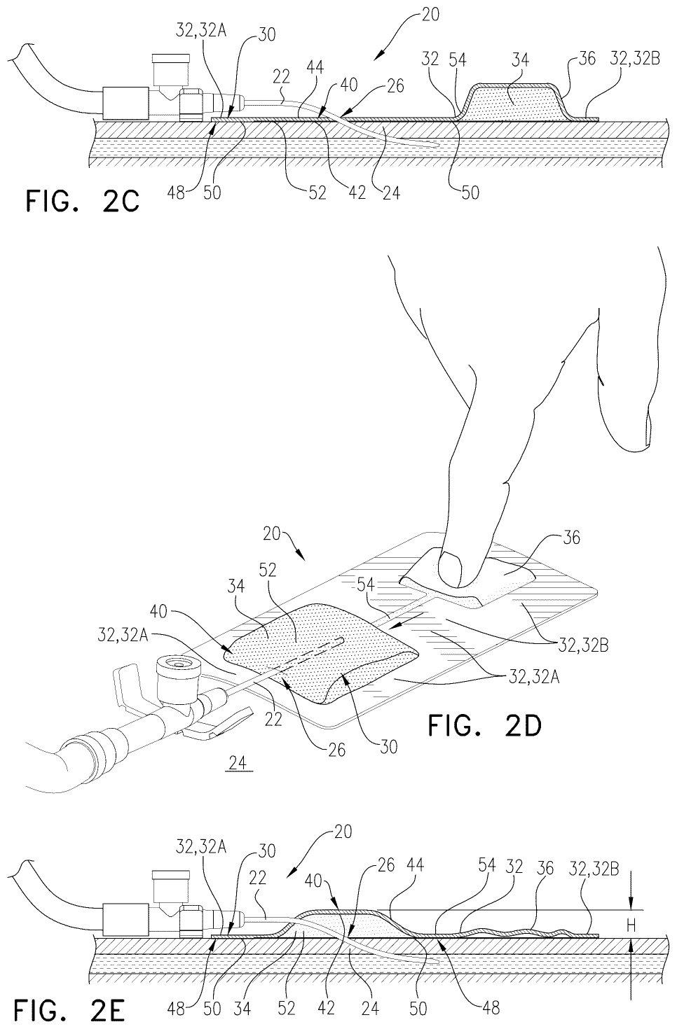

FIGS. 1A and 1B are schematic isometric and cross-sectional views of a dressing 20, in accordance with an application of the present invention. Dressing 20 is for use with a percutaneous medical device 22 for insertion through skin 24 of a patient at a skin-insertion site 26, as described hereinbelow with reference to FIGS. 2A-E.

Percutaneous medical device 22 may comprise any medical device that is disposed partially within the body and partially exposed outside the body, and passes through a disruption in the skin. For example, the percutaneous medical device may comprise a percutaneous-insertion instrument, such as a catheter, or an orthopedic percutaneous fixation device, such as a percutaneous pin.

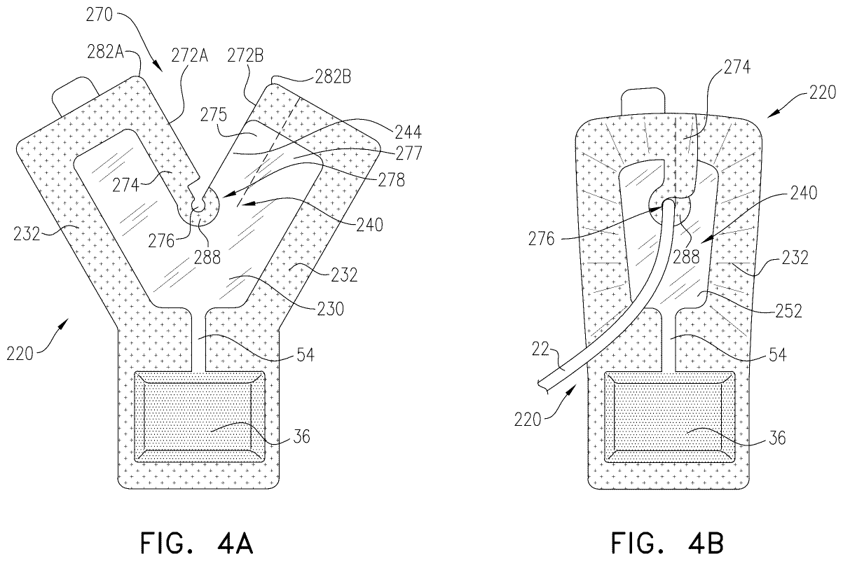

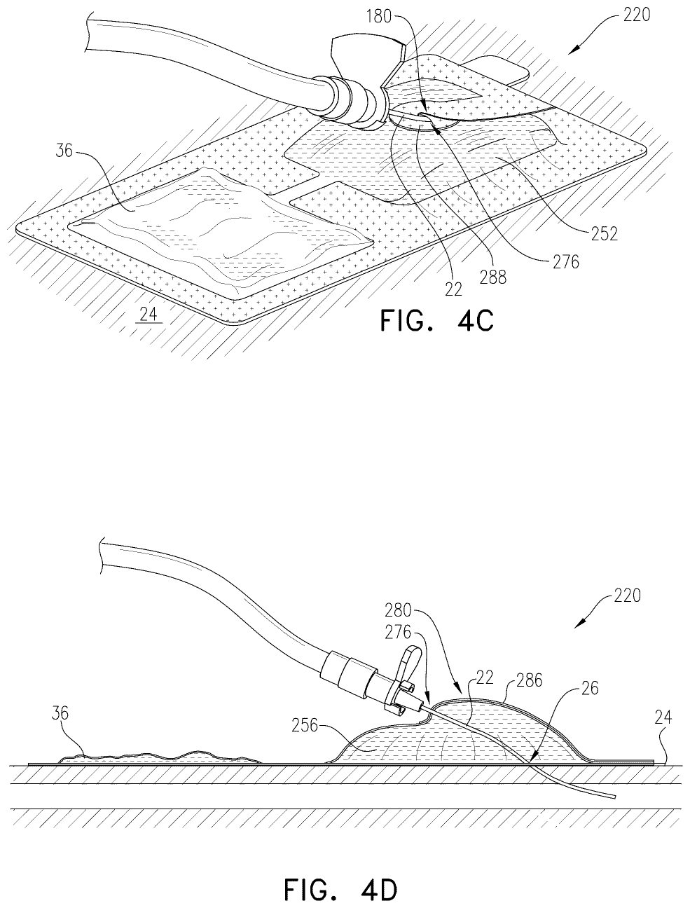

Dressing 20 comprises an upper layer 30, an adhesive area 32, a flowable substance 34, and one or more reservoirs 36 containing flowable substance 34. For example, in any of the configurations of dressings described herein as comprising one or more reservoirs 36, the dressing may comprise exactly one reservoir 36. Optionally, the one or more reservoirs 36 are disposed to the side of insertion area 40, such as shown. Upper layer 30 comprises a thin sheet of material, such as a polyurethane film substrate. Upper layer 30 defines an insertion area 40 having a non-adhesive skin-facing surface 42 (labeled in FIGS. 2D and 2E) configured to be positioned above skin-insertion site 26. Upper layer 30 is configured to allow passage of percutaneous medical device 22 through insertion area 40, for example by puncturing of upper layer 30, such as described hereinbelow with reference to FIGS. 2A-E, or via an opening through insertion area 40, such as described hereinbelow with reference to FIGS. 4A-D. Typically, an upper surface 44 (labeled in FIGS. 2C and 2E) of upper layer 30 is non-adhesive. Optionally, dressing 20 comprises one or more additional layers over upper layer 30 (configuration not shown). Insertion area 40 is typically defined by a central area of upper layer 30.

For some applications, insertion area 40 of upper layer 30 is transparent, such as to enable visual inspection of skin-insertion site 26.

Although upper layer 30 is shown in FIGS. 1A-B as rectangular with rounded corners, the upper layer may have any other convenient shape, such as elliptical, circular, polygonal (e.g., square, triangular, trapezoidal, or rectangular), or the shapes described hereinbelow with reference to FIGS. 4A-D.

For some applications, flowable substance 34 comprises a gel or a cream (such as any known antimicrobial ointment and/or a hydrogel). For other applications, flowable substance 34 comprises a liquid.

For some applications, flowable substance 34 comprises an antimicrobial agent (e.g., an antibacterial) and/or an antiseptic agent.

For other applications, flowable substance 34 does not comprise an antimicrobial agent (e.g., an antibacterial) or an antiseptic agent. Instead, non-adhesive skin-facing surface 42 of insertion area 40 is coated with a solid, gel, or liquid antimicrobial agent (e.g., an antibacterial) and/or an antiseptic agent. Upon flowing of flowable substance 34 from the one or more reservoirs 36 to insertion area 40, as described below, flowable substance 34 combines with the antimicrobial and/or antiseptic agent to produce a liquid or gel antimicrobial and/or antiseptic within fillable insertion chamber 52, described hereinbelow. For example, the antimicrobial and/or antiseptic agent may dissolve in and/or mix with flowable substance 34.

For some applications, flowable substance 34 comprises a glue, which may help provide good fixation for percutaneous medical device 22, particularly in applications in which percutaneous medical device 22 comprises a percutaneous-insertion instrument. For some of these applications, flowable substance 34 comprises an antimicrobial agent and/or an antiseptic agent, disposed in the glue. The glue may comprise an epoxy, as described hereinbelow.

For some applications, flowable substance 34 is configured to change color upon the occurrence of one or more of the following:
microbial contamination, which may enable easy identification of infection,
a change in a pH of flowable substance 34 (e.g., by a certain predetermined amount),
a change in a chemical configuration of flowable substance 34, and/or
exposure of flowable substance 34 to blood, reflecting bleeding at skin-insertion site 26.

Adhesive area 32 has a skin-facing surface 48 coated with an adhesive 50 (labeled in FIGS. 2D and 2E). A peripheral portion of adhesive area 32 is disposed around at least a portion of a perimeter 46 of insertion area 40 (labeled in FIG. 1A), such as around at least 70%, at least 90%, or at least 95% of perimeter 46 of insertion area 40. For some applications, adhesive area 32 entirely surrounds perimeter 46 of insertion area 40, and upper layer 30 is configured to allow passage of percutaneous medical device 22 through insertion area 40 by puncturing of upper layer 30.

Insertion area 40 of upper layer 30 is shaped so as to define a fillable insertion chamber 52 between non-adhesive skin-facing surface 42 and skin 24 when dressing 20 is adhered to skin 24 by at least a portion of adhesive area 32, such as described hereinbelow with reference to FIGS. 2A-E. For some applications, insertion area 40 of upper layer 30 is flexible, and optionally clastic. For some applications, insertion area 40 of upper layer 30 is pre-shaped so as to define fillable insertion chamber 52. For some applications in which insertion area 40 is not pre-shaped so as to define fillable insertion chamber 52, when dressing 20 is initially adhered to skin 24, fillable insertion chamber 52 does not define an internal volume, because insertion area 40 is placed snugly against skin 24 (without adhering to the skin). For other applications in which insertion area 40 is not pre-shaped so as to define fillable insertion chamber 52, fillable insertion chamber 52 defines an internal volume upon initial application of dressing 20 to skin 24, such as described, for example, hereinbelow with reference to FIGS. 4A-D.

The one or more reservoirs 36 are separate from fillable insertion chamber 52, and are connected to insertion area 40 by one or more passages 54 (e.g., exactly one passage 54) so as to enable flow of flowable substance 34 from the one or more reservoirs 36 to insertion area 40 (and fillable insertion chamber 52) upon application of pressure to the one or more reservoirs 36. The pressure is typically applied to an external surface of the one or more reservoirs 36, such as manually (e.g., by a finger of a healthcare worker); alternatively, the pressure may be applied to an interior of the one or more reservoirs 36 (e.g., by filling one or more balloons within the one or more reservoirs 36). Further alternatively, one or more walls of the one or more reservoirs 36 may be configured to apply the pressure to the one or more reservoirs 36, such as after an initial small amount of pressure is applied to an external surface of the one or more reservoirs 36, such as to break an internal seal; for example, the one or more walls may comprise an elastic material.

For some applications, the peripheral portion of adhesive area 32 entirely surrounds perimeter 46 of insertion area 40 except at one or more locations 56 (labeled in FIG. 1A) at which the one or more passages 54 connect to insertion area 40, and upper layer 30 is configured to allow passage of percutaneous medical device 22 through insertion area 40 by puncturing of upper layer 30.

For some applications, dressing 20 comprises a removable protective backing 60 (labeled in FIG. 1B) that removably covers (a) non-adhesive skin-facing surface 42 of insertion area 40 and (b) adhesive area 32.

For some applications, adhesive area 32 is a first adhesive area 32, 32A, and dressing 20 comprises a second adhesive area 32, 32B that is configured to adhere the one or more reservoirs 36 to skin 24. This may allow squeezing of the reservoirs without applying any pressure to skin 24, such as before adhering the one or more reservoirs 36 to skin 24. For some of these applications, removable protective backing 60 removably covers (a) non-adhesive skin-facing surface 42 of insertion area 40, (b) first adhesive area 32, 32A, and (c) second adhesive area 32, 32B.

For some applications, upper layer 30 defines, in addition to insertion area 40, a peripheral area 64, which surrounds at least 50% (e.g., at least 70%, 80%, 90%, or 95%) of perimeter 46 of insertion area 40, and which typically defines skin-facing surface 48 coated with adhesive 50. For some of these applications, dressing 20 further comprises a peripheral reinforcement, which is coupled to and reinforces peripheral area 64 of upper layer 30 (configuration not shown). For some applications, the peripheral reinforcement defines skin-facing surface 48 coated with adhesive 50.

For some applications, dressing 20 further comprises a peripheral reinforcement, which surrounds at least 50% (e.g., at least 70%, 80%, 90%, or 95%) of perimeter 46 of insertion area 40 (configuration not shown). For some of these applications, the peripheral reinforcement defines skin-facing surface 48 coated with adhesive 50.

For some of these applications, adhesive area 32 is disposed around at least 70%, at least 90%, at least 95%, or an entirety of perimeter 46 of insertion area 40.

For some applications, flowable substance 34 has a volume greater than the product of (a) 1 mm (e.g., 2 mm, 3 mm, 5 mm, or 10 mm) and (b) an area of insertion area 40 of upper layer 30. Alternatively or additionally, for some applications, a volume of flowable substance 34 is at least 0.1 cc, e.g., at least 0.25 cc, such as at least 1 cc, e.g., at least 2 cc, at least 3 cc, at least 5 cc, or at least 10 cc.

For some applications, an area of insertion area 40 of upper layer 30 is at least 0.5 cm2, such as at least 1 cm2 or at least 2 cm2, and, optionally, no more than 25 cm2, such as no more than 15 cm2.

For some applications, dressing 20 is configured such that when adhesive area 32 is adhered to skin 24 and fillable insertion chamber 52 is filled with 60% of flowable substance 34 from the one or more reservoirs 36, a greatest height H of flowable substance 34 within fillable insertion chamber 52 is at least 1 mm, such as at least 2 mm, at least 3 mm, at least 5 mm, or at least 10 mm (labeled in FIG. 2C, described hereinbelow).

For some applications of the present invention, a kit is provided that comprises dressing 20 and percutaneous medical device 22.

For some applications, flowable substance 34 comprises a liquid hardener of an epoxy, and a solid resin (e.g., in powder form) of the epoxy coats non-adhesive skin-facing surface 42 of insertion area 40. For some of these applications, one or both of the liquid hardener and the solid resin comprise an antimicrobial agent and/or an antiseptic agent.

For some applications, flowable substance 34 comprises a liquid resin of an epoxy, and a solid hardener (e.g., in powder form) of the epoxy coats non-adhesive skin-facing surface 42 of insertion area 40. For some of these applications, one or both of the liquid resin and the solid hardener comprise an antimicrobial agent and/or an antiseptic agent.

For some applications, dressing 20 further comprises a middle absorbent layer disposed between non-adhesive skin-facing surface 42 and upper layer 30 (configuration not shown).

Reference is now made to FIGS. 2A-E, which are schematic illustrations of a method using dressing 20 of FIGS. 1A-B, in accordance with an application of the present invention. FIGS. 2A, 2B, and 2D are isometric views, and FIGS. 2C and 2E are cross-sectional side views of FIGS. 2B and 2D, respectively.

As shown in FIG. 2A, dressing 20 is applied to skin 24 by positioning, above skin-insertion site 26, non-adhesive skin-facing surface 42 of insertion area of upper layer 30 of dressing 20. At least a portion of skin-facing surface 48 of adhesive area 32 is adhered to skin 24, such that insertion area 40 is shaped so as to define fillable insertion chamber 52 between non-adhesive skin-facing surface 42 and skin 24. For applications in which dressing 20 further comprises removable protective backing 60, before the at least a portion of skin-facing surface 48 of adhesive area 32 is adhered to skin 24, removable protective backing 60 is removed.

As shown in FIGS. 2B and 2C, percutaneous medical device 22 is caused to pass through insertion area 40 and is passed through skin 24 at skin-insertion site 26. For example, percutaneous medical device 22 may be passed through insertion area 40 by puncturing upper layer 30, as shown in FIGS. 2B and 2C. Alternatively, percutaneous medical device 22 may be passed through insertion area such as described hereinbelow with reference to FIGS. 4A-D. For applications in which percutaneous medical device 22 comprises a percutaneous medical device, such as a catheter, the same needle that is normally used to inserted the catheter through the skin is used to penetrate upper layer 30. The fixability of upper layer 30 ensures the seal of the penetration point of the catheter through upper layer 30.

Although percutaneous medical device 22 is shown in FIG. 2B as penetrating skin 24 from outside the body to inside the body, percutaneous medical device 22 may alternatively penetrate the skin in the opposite direction, such as during installation of a medical device in which the medical device is inserted into the body via some other route and a portion of the device is passed out of the body via the skin-insertion site (in which case, percutaneous medical device 22 typically passes through skin 24 before passing through the insertion area). As used in the present application, including in the claims, the term “skin-insertion site” refers to the site of the skin at which percutaneous medical device 22 passes through the skin, regardless of which direction the device was passed through the skin. Similarly, the phrase “insertion through skin” is to be understood as insertion through skin either from outside the body to inside the body, or from inside the body to outside the body.

Although in the sequences of steps illustrated with reference to FIGS. 2A-C percutaneous medical device 22 is passed through insertion area 40 after dressing 20 has been applied to skin 24, alternatively percutaneous medical device 22 may be passed through insertion area 40 while applying dressing 20 to skin 24, for example using dressing 220 or 320, described hereinbelow with reference to FIGS. 4A-D and 8, respectively.

As shown in FIGS. 2D and 2E, after dressing 20 is applied to skin 24 (and optionally after percutaneous medical device 22 has been passed through insertion area 40 and through skin 24 at skin-insertion site 26), pressure is applied to one or more reservoirs 36 of dressing 20, such that flowable substance 34 contained within the one or more reservoirs 36 flows from the one or more reservoirs 36 to fillable insertion chamber 52 via the one or more passages 54 connecting the one or more reservoirs 36 and insertion area 40. This flowing of flowable substance 34 into fillable insertion chamber 52 typically results in homogeneous distribution of the flowable substance within the fillable insertion chamber and homogeneous contact with both skin 24 and the portion of percutaneous medical device 22 that is inside the fillable insertion chamber.

As mentioned above, in some applications insertion area 40 of upper layer 30 is elastic. In some of these applications, insertion area 40 may touch (e.g., be placed snugly against) skin 24 (without adhering to the skin) upon application of dressing 20 to skin 24, as shown in FIG. 2A, and during passage of percutaneous medical device 22 through insertion area 40 and through skin 24 at skin-insertion site 26, as shown in FIGS. 2B and 2C. Thus, during this stage of the method, fillable insertion chamber 52 may optionally not define an internal volume, or may substantially not define an internal volume. For other applications, fillable insertion chamber 52 defines an internal volume upon initial application of dressing 20 to skin 24, such as described, for example, hereinbelow with reference to FIGS. 4A-D.

For some applications, applying the pressure causes flowable substance 34 to flow to fillable insertion chamber 52 such that the greatest height H of flowable substance 34 within fillable insertion chamber 52 is at least 1 mm, such as at least 2 mm, at least 3 mm, at least 5 mm, or at least 10 mm (labeled in FIG. 2C).

For some applications, when adhesive area 32 is adhered to skin 24, such as shown in FIGS. 2A-E, the one or more passages 54 are open to skin 24 such that skin 24 partially defines the one or more passages 54.

For some applications, the one or more passages 54 are configured to inhibit (e.g., prevent) the flow of flowable substance 34 from the one or more reservoirs 36 to insertion area 40 when one or more respective interiors of the one or more reservoirs 36 are at atmospheric pressure (i.e., before application of the pressure). For some of these applications the one or more passages 54 are not valved, while for others of these applications, the one or more passages 54 comprise respective valves, such as one-way valves, e.g., duckbill valves.

For some applications, the one or more passages 54 are configured to inhibit (e.g., prevent) backflow of flowable substance 34 from fillable insertion chamber 52 back to the one or more reservoirs 36. For example, the one or more passages 54 may comprise respective valves, such as one-way valves, e.g., duckbill valves.

For some applications in which dressing 20 further comprises removable protective backing 60, the removable protective backing removably covers (a) non-adhesive skin-facing surface 42 of insertion area 40, (b) adhesive area 32, and (c) at least a portion of the one or more passages 54, so as to seal the one or more passages 54. For some of these applications, a portion of an upper surface of removable protective backing 60 that removably covers the at least a portion of the one or more passages 54 comprises an adhesive that removably adheres to an upper wall of the at least a portion of the one or more passages 54, so as to seal the one or more passages 54.

Reference is still made to FIGS. 2A-E. For some applications, such as in configurations in which dressing 20 comprises exactly one reservoir 36, reservoir 36 is used to initially fill fillable insertion chamber 52 with a first portion of flowable substance 34 upon application of dressing 20 to skin 24, and to later refill fillable insertion chamber 52 with a second portion of fresh flowable substance 34, typically after the first portion of the flowable substance has at least partially drained from fillable insertion chamber 52, e.g., as a result of leakage or absorption by skin 24, or of active draining by a healthcare worker. This technique may reduce the number of times that dressing 20 needs to be changed. For some applications, the first portion of flowable substance 34 is at least partially drained from fillable insertion chamber 52 while dressing 20 remains at least partially applied to skin 24 (one edge may need to be released from skin 24 to provide an opening for drainage of the first portion of flowable substance 34, or dressing 20 may remain entirely applied to skin 24, and the first portion of flowable substance 34 is at least partially drained from fillable insertion chamber 52 using a syringe). Thereafter, pressure is again applied to reservoir 36 such that the second portion of flowable substance 34 flows from reservoir 36 to fillable insertion chamber 52.

To this end, an appropriate volume of flowable substance 34 is provided to provide both the first and the second portions of flowable substance 34. For example, a volume of flowable substance 34 in reservoir 36 may be greater than the product of (a) 2 and (b) 1 mm (e.g., 2 mm, 3 mm, 5 mm, or 10 mm) and (c) an area of insertion area 40 of upper layer 30.

The techniques described in the two immediately preceding paragraphs may optionally be implemented in any of the dressings described herein.

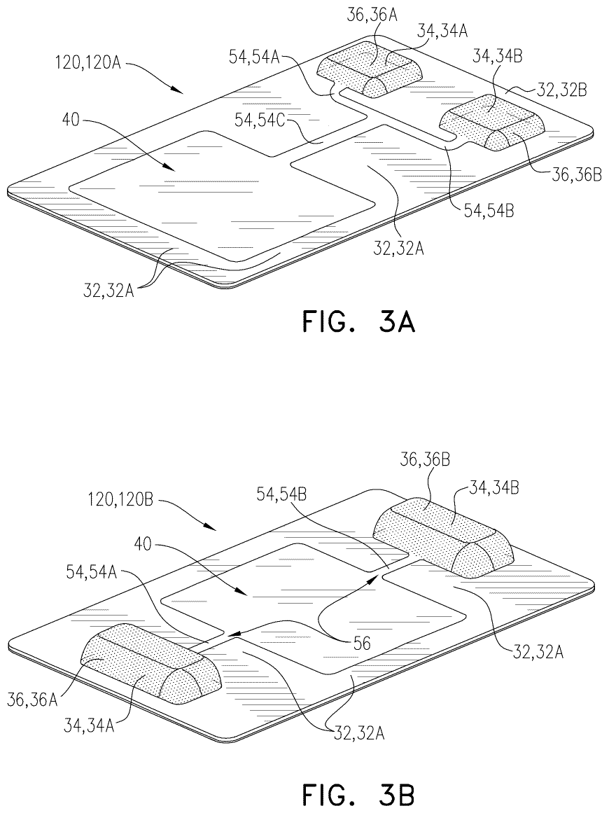

Reference is now made to FIGS. 3A and 3B, which are schematic isometric views of two configurations of dressing 120, in accordance with respective applications of the present invention. Other than as described below, dressing 120 is identical to dressing 20, described hereinabove with reference to FIGS. 1A-2E, and may implement any of the techniques of dressing 20 described hereinabove. Like reference numerals refer to like parts.

In these configurations, dressing 120 comprises first and second reservoirs 36A and 36B, which contain respective first portion 34A and second portion 34B of flowable substance 34. Dressing 20 further comprises first and second passages 54A and 54B, which connect first and second reservoirs 36A and 36B to insertion area 40, respectively. First and second passages 54A and 54B may merge into a common passage 54C, which is connected to insertion area 40, such as shown for dressing 120, 120A in FIG. 3A. Alternatively, first and second passages 54A and 54B may be separately connected to insertion area 40, such as shown for dressing 120, 120B in FIG. 3B. In this latter configuration, first and second reservoirs 36A and 36B may optionally be disposed on different sides of insertion area 40, such as at opposite sides of the insertion area, as shown in FIG. 3B. Optionally, dressing 120 comprises three or more reservoirs 36.

During use of dressing 120, pressure is applied to first and second reservoirs 36A and 36B such that respective first and second portions 34A and 34B of flowable substance 34 flow from first and second reservoirs 36A and 36B, respectively, to fillable insertion chamber 52.

For some applications, first and second reservoirs 36A and 36B are separately used to initially fill fillable insertion chamber 52 with first portion 34A of flowable substance 34 upon application of dressing 120 to skin 24, and to later refill fillable insertion chamber 52 with second portion 34B of fresh flowable substance 34, typically after the first portion of the flowable substance has at least partially drained from fillable insertion chamber 52, e.g., as a result of leakage or absorption by skin 24, or of active draining by a healthcare worker. This technique may reduce the number of times that dressing 120 needs to be changed. In this technique, pressure is applied to first reservoir 36A, and not to second reservoir 36B, such that first portion 34A of flowable substance 34 flows from first reservoir 36A to fillable insertion chamber 52. Thereafter, for some applications, first portion 34A of flowable substance 34 is at least partially drained from fillable insertion chamber 52 while dressing 120 remains at least partially applied to skin 24 (one edge may need to be released from skin 24 to provide an opening for drainage of first portion 34A, or dressing 120 may remain entirely applied to skin 24, and first portion 34A of flowable substance 34 is at least partially drained from fillable insertion chamber 52 using a syringe). Thereafter, pressure is applied to second reservoir 36B such that second portion 34B of flowable substance 34 flows from second reservoir 36B to fillable insertion chamber 52.

To this end, appropriate volumes of first and second portions 34A and 34B of flowable substance 34 are provided. For example, a volume of first portion 34A of flowable substance 34 in first reservoir 36A may be greater than the product of (a) 1 mm (e.g., 2 mm, 3 mm, 5 mm, or 10 mm) and (b) an area of insertion area 40 of upper layer 30; and a volume of second portion 34B of flowable substance 34 in second reservoir 36B may be greater than the product of (a) 1 mm (e.g., 2 mm, 3 mm, 5 mm, or 10 mm) and (b) an area of insertion area 40 of upper layer 30.

For some applications, first and second reservoirs 36A and 36B are both used to initially fill fillable insertion chamber 52 with both first and second portions 34A and 34B of flowable substance 34 upon application of dressing 120 to skin 24, rather than waiting between fillings of the first and the second portions.

For some of these applications, flowable substance 34 comprises an epoxy. First portion 34A of flowable substance 34 comprises a liquid resin of the epoxy, and second portion 34B of flowable substance 34 comprises a liquid hardener of the epoxy (a hardener is also known as a curing agent in the epoxy art). For some applications, an antimicrobial agent (e.g., an antibacterial agent) and/or an antiseptic agent is disposed within at least one of the liquid resin and the liquid hardener.

During use of dressing 120, pressure is applied to first and second reservoirs 36A and 36B such that the liquid resin and the liquid hardener flow from first and second reservoirs 36A and 36B, respectively, to fillable insertion chamber 52 and mix in fillable insertion chamber 52. The epoxy, upon hardening, secures and fixates the percutaneous medical device 22 in position. By applying adhesive to fillable insertion chamber 52, after the adhesive is cured the catheter fixation is improved.

Reference is now made to FIGS. 4A-D, which are schematic isometric views of a dressing 220, in accordance with an application of the present invention. Other than as described below, dressing 220 is identical to dressing 20, described hereinabove with reference to FIGS. 1A-2E. Dressing 220 may implement any techniques of dressing 20 described hereinabove and/or of dressing 120, described hereinabove with reference to FIGS. 3A-B. Like reference numerals refer to like parts.

FIG. 4A shows dressing 220 prior to application to skin 24, and FIGS. 4B-D show dressing 220 after application to skin 24. As can be seen in FIG. 4A, an insertion area 240 defined by an upper layer 230 of dressing 220 is shaped so as to define a slit 270 having first and second edges 272A and 272B. An adhesive-area slit-closing portion 274 of an adhesive area 232 of dressing 220 is disposed alongside at least first edge 272A of slit 270, and is configured to be adhered to an upper surface 277 of an upper-layer slit-closing portion 275 of upper layer 230 alongside second edge 272B of slit 270, as shown in FIGS. 4B-D. As a result, adhesive-area slit-closing portion 274 of adhesive area 232 and upper-layer slit-closing portion 275 of upper layer 230 overlap alongside first and second edges 272A and 272B. For example, the overlap may have an average width of 0.3 cm.

Typically, as shown in FIGS. 4B-D, upper layer 230 is configured to allow passage of percutaneous medical device 22 through insertion area 240 via an opening 276 through insertion area 240 defined at a junction 278 of respective inner ends of first and second edges 272A and 272B of slit 270, respectively, as labeled in FIG. 4A. (The inner ends are “inner” in the sense that they are not peripheral ends 282A and 282B of first and second edges 272A and 272B.)

During application of dressing 120, adhesive-area slit-closing portion 274 of adhesive area 232 is adhered to upper surface 277 of upper-layer slit-closing portion 275 of upper layer 230 alongside second edge 272B of slit 270, as shown in FIGS. 4B-D.

For some applications, dressing 220 is shaped such that application of adhesive-area slit-closing portion 274 of adhesive area 232 to upper surface 277 of upper-layer slit-closing portion 275 of upper layer 230 forms fillable insertion chamber 252. For some of these applications, dressing 220 is shaped such that application of adhesive-area slit-closing portion 274 of adhesive area 232 to upper surface 277 of upper-layer slit-closing portion 275 of upper layer 230 provides a tented shape 284 to a fillable insertion chamber 252 defined by insertion area 240 when dressing 220 is adhered to skin 24. Tented shape 284 typically defines a single apex 280 from which walls 286 of fillable insertion chamber 252 slope downward toward skin 24, optionally such that tented shape 284 is an irregular conical or irregular frustoconical shape, somewhat similar to the conical shape of a paper cone formed by adhering together the two straight edges of a portion of a circular piece of paper, as is known in the crafts art. Typically, junction 278 is located at apex 280 of tented shape 284, such that percutaneous medical device 22 passes through insertion area 240 at or near apex 280.

Typically, a skin-facing surface of upper-layer slit-closing portion 275 of upper layer 230 is non-adhesive, such that upper-layer slit-closing portion 275 can move away from skin 24 during the formation of fillable insertion chamber 252 by application of adhesive-area slit-closing portion 274 of adhesive area 232 to upper surface 277 of upper-layer slit-closing portion 275. By contrast, if the skin-facing surface of upper-layer slit-closing portion 275 of upper layer 230 were adhesive, as in some convention U-shaped adhesive dressings, adhesion of the skin-facing surface of upper-layer slit-closing portion 275 to skin 24 would inhibit, and possibly prevent, movement of skin-closing portion 275 away from skin 24, such that dressing 220 could not form fillable insertion chamber 252.

For some applications, percutaneous medical device 22 is passed through insertion area 240 via opening 276 after percutaneous medical device 22 has been passed through skin 24 at skin-insertion site 26 (either from outside the skin to inside the body, or vice versa, as described hereinabove). For some of these applications, percutaneous medical device 22 is passed through insertion area 240 via opening 276 by positioning first and second edges 272A and 272B of slit 270 around a longitudinal portion of percutaneous medical device 22 such that percutaneous medical device 22 passes through opening 276.

For some applications, slit 270 is wedge-shaped, such as shown in FIG. 4A.

For some applications, dressing 220 is Y-shaped, such as shown in FIG. 4A, or U-shaped (configuration not shown).

For some applications, a percutaneous-medical-device-adhering portion 288 of adhesive area 232 is disposed on a skin-facing surface of insertion area 240 at least partially surrounding opening 276 at junction 278. Percutaneous-medical-device-adhering portion 288 is configured to adhere to an external surface of percutaneous medical device 22 when percutaneous medical device 22 is disposed passing through opening 276, thereby helping fixate percutaneous medical device 22 with respect to dressing 220 and skin-insertion site 26.

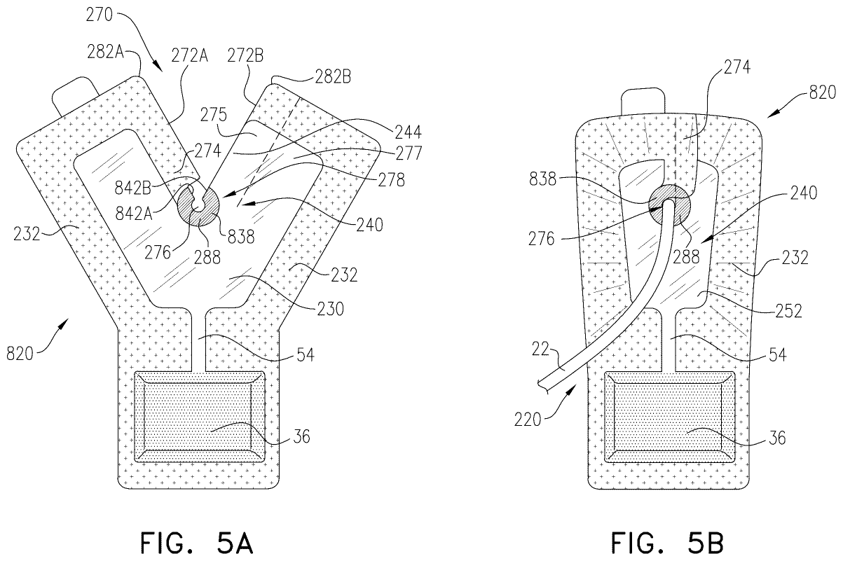

Reference is made to FIGS. 5A-B, which are schematic illustrations of a dressing 820, in accordance with an application of the present invention. Other than as described below, dressing 820 is identical to dressing 220, described hereinabove with reference to FIGS. 4A-D, and may implement any of the features thereof, mutatis mutandis. Like reference numerals refer to like parts. FIG. 5A shows dressing 820 prior to application to skin 24, and FIG. 5B shows dressing 820 after application to skin 24.

Dressing 820 further comprises a seal 838 shaped so as to define a seal slit 840 having first and second seal-slit edges 842A and 842B. Seal 838 has properties (e.g., material properties or thickness) different in at least one respect from those of material of a remainder of insertion area 240 of upper layer 230. For example, seal 838 may comprise a polymer (e.g., an elastomer), such as silicone, rubber, or synthetic material (e.g., silicone rubber synthetic material), in which case seal 838 may be a septum seal, as known in the drug delivery art. Alternatively or additionally, for example, seal 838 may have a thickness greater than a thickness of the material of a remainder of insertion area 240 of upper layer 230, in which case seal 838 may comprise the same as, or a different material from, the material of a remainder of insertion area 240 of upper layer 230. Using a thinner and/or more flexible material for the remainder of insertion area 240 of upper layer 230 may help ensure that the insertion area as a whole remains highly flexible.

Seal 838 is coupled to insertion area 240 at least partially surrounding opening 276 at junction 278. Seal 838 is configured to form a tight seal around percutaneous medical device 22 when device 22 is passed through the seal and opening 276 through insertion area 240, and adhesive-area slit-closing portion 274 of adhesive area 232 is adhered to upper surface 277 of upper-layer slit-closing portion 275 of upper layer 230 alongside second edge 272B of slit 270 of insertion area 240.

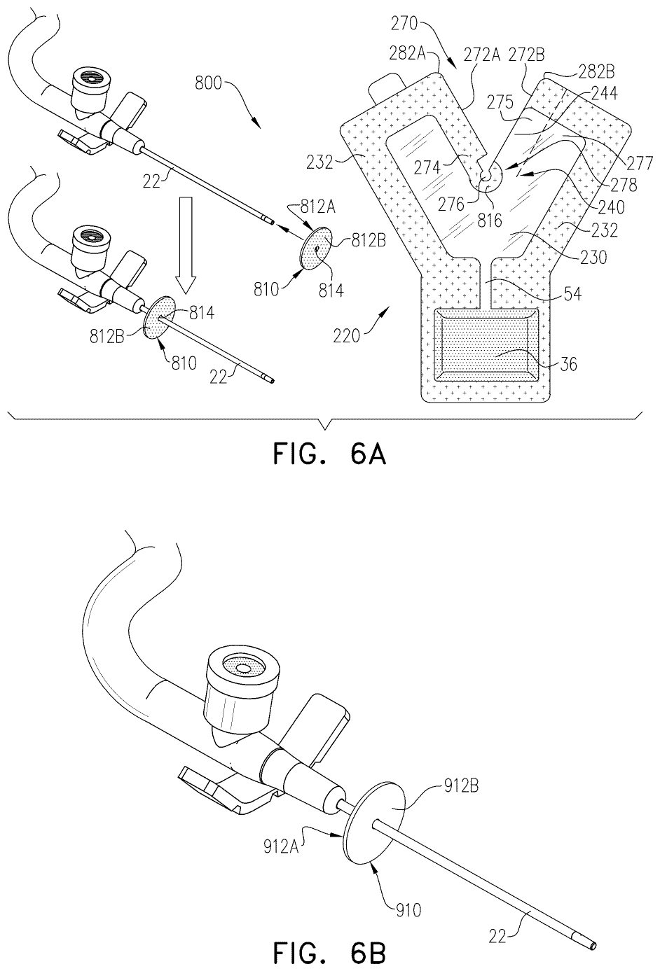

Reference is now made to FIG. 6A, which is a schematic illustration of a kit 800, in accordance with an application of the present invention. Kit 800 comprises dressing 220, described hereinabove with reference to FIGS. 4A-D (and optionally with reference to FIGS. 5A-B). Kit 800 further comprises a sealing support 810. Kit 800 may or may not comprise percutaneous medical device 22.

Sealing support 810 is shaped so as to define (a) an upper surface 812A. (b) a lower surface 812B and (c) a hole 814, which passes through sealing support 810 between upper and lower surfaces 812A and 812B. Hole 814 is configured to form a tight seal around percutaneous medical device 22 when percutaneous medical device 22 is disposed passing through hole 814. For example, sealing support 810 may comprise a polymer (e.g., an elastomer), such as silicone, rubber, or synthetic material (e.g., silicone rubber synthetic material), in which case scaling support 810 may be similar to a septum seal, as known in the drug delivery art.

Optionally, sealing support 810 is circular, i.e., shaped as a disc.

Typically, at least upper surface 812A of sealing support 810 is flat when scaling support 810 is in a relaxed state without external force applied thereto.

For some applications, upper surface 812A of sealing support 810 has a surface area of at least 19 mm2, no more than 85 mm2, and/or 19-85 mm2, and/or a diameter of at least 5 mm, no more than 10 mm, and/or 5-10 mm.

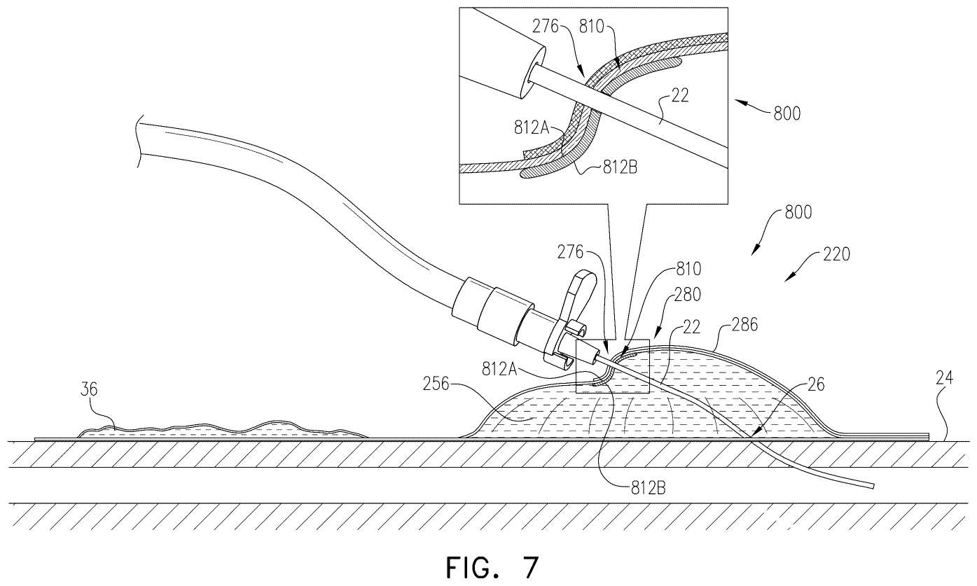

A scaling-support-adhering portion 816 of adhesive area 232 is disposed on a skin-facing surface of insertion area 240 at least partially surrounding opening 276 at junction 278. Sealing-support-adhering portion 816 is configured to adhere to upper surface 812A of sealing support 810 when percutaneous medical device 22 is disposed passing through hole 814 of sealing support 810 and opening 276 through insertion area 240 defined by upper layer 230 of dressing 220, such as described hereinbelow with reference to FIG. 7.

Reference is now made to FIG. 6B, which is a schematic illustration of a kit 900, in accordance with an application of the present invention. Kit 900 comprises dressing 220, described hereinabove with reference to FIGS. 4A-D (and optionally with reference to FIGS. 5A-B); dressing 220 is not shown in FIG. 6B. Kit 900 further comprises a percutaneous medical device 922, which may implement any of the features of percutaneous medical device 22 described herein.

Percutaneous medical device 922 comprises a sealing support 910, which may be fabricated as an integral portion of percutaneous medical device 922, or as a separate piece that is coupled to percutaneous medical device 922, typically during manufacture. Sealing support 910 is shaped so as to define an upper surface 912A and a lower surface 912B. Percutaneous medical device 922 is disposed passing through sealing support 910 such that scaling support 910 surrounds a portion of percutaneous medical device 922.

Optionally, sealing support 910 implements any of the features of sealing support 810, described hereinabove with reference to FIG. 6A.

Sealing-support-adhering portion 816 of adhesive area 232 is disposed on a skin-facing surface of insertion area 240 at least partially surrounding opening 276 at junction 278, such as shown above for kit 800 in FIG. 6A.

Sealing-support-adhering portion 816 is configured to adhere to upper surface 912A of sealing support 910 when percutaneous medical device 922 is disposed passing through opening 276 through insertion area 240 defined by upper layer 230 of dressing 220, such as described hereinbelow with reference to FIG. 7 for kit 800.

Reference is now made to FIG. 7, which is a schematic illustration of dressing 220 of kit 800, described hereinabove with reference to FIG. 6A, after application to skin 24, in accordance with an application of the present invention. Dressing 220 of kit 900, described hereinabove with reference to FIG. 6B, may be applied to skin 24 in the same manner.

To use kit 800, before causing percutaneous medical device 22 to pass through insertion area 240, percutaneous medical device 22 is disposed passing through hole 814 defined by sealing support 810 such that hole 814 forms a tight seal around percutaneous medical device 22, such as shown in FIG. 6A.

Causing percutaneous medical device 22 to pass through insertion area 240 via opening 276 comprises adhering sealing-support-adhering portion 816 to upper surface 812A of sealing support 810.

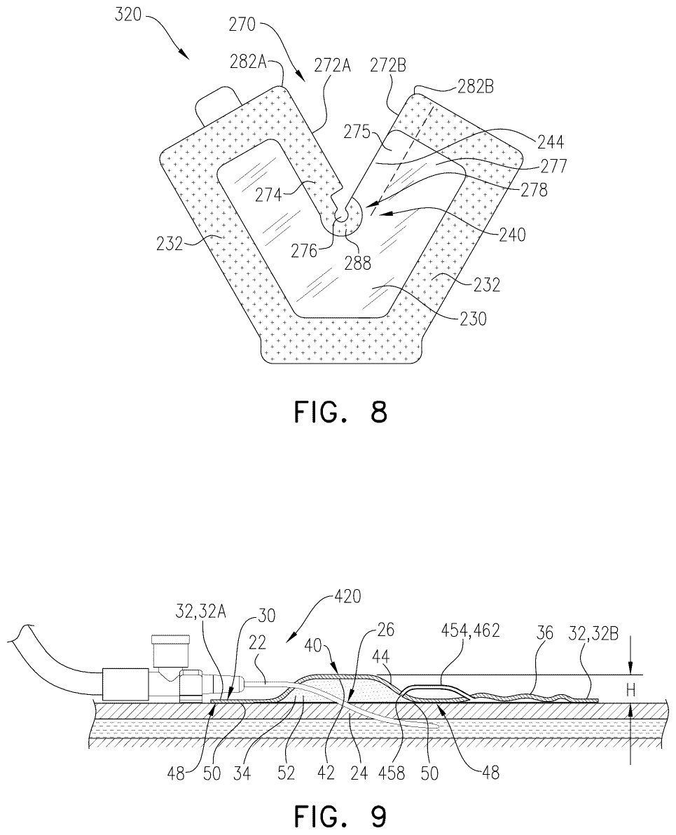

Reference is made to FIG. 8, which is a schematic illustration of a dressing 320, in accordance with an application of the present invention. Other than as described below, dressing 320 is identical to dressing 220, described hereinabove with reference to FIGS. 4A-D, and may implement any of the features thereof, as well as the other dressing described herein, mutatis mutandis. Unlike dressing 220, dressing 320 does not comprise any reservoirs 36 or flowable substance 34. Instead, a flowable substance is typically introduced into fillable insertion chamber 252, such as using a conventional syringe, after adhesion of dressing 320 to skin 24.

Reference is now made to FIG. 9, which is a schematic illustration of a dressing 420, in accordance with an application of the present invention. Other than as described below, dressing 420 is identical to dressing 20, described hereinabove with reference to FIGS. 1A-2E, and may implement any of the features thereof, mutatis mutandis. The features of dressing 420 may also optionally be implemented in combination with any of the other dressings described herein.

In the configuration shown in FIG. 9, the one or more reservoirs 36 are connected to insertion area 40 by one or more passages 454 via one or more openings 458 through insertion area 40 of upper layer 30; for example, the one or more passages 454 may comprise one or more respective external tubes 462 in this configuration.

Reference is now made to FIG. 10, which is a schematic isometric view of a dressing 520, in accordance with an application of the present invention. Other than as described below, dressing 520 is identical to dressing 20, described hereinabove with reference to FIGS. 1A-2E, and may implement any of the techniques of dressing 20 described hereinabove and/or the other dressings described herein, mutatis mutandis. Likewise, dressing 20 and the other dressings described herein may optionally implement any of the techniques of dressing 520, mutatis mutandis. Like reference numerals refer to like parts. Dressing 520 is for use with percutaneous medical device 22 for insertion through skin 24 of a patient at skin-insertion site 26, as described hereinabove for dressing 20 with reference to FIGS. 2A-E, mutatis mutandis.

Dressing 520 comprises an upper layer 530, which defines an insertion area 540 having non-adhesive skin-facing surface 42 configured to be positioned above skin-insertion site 26; these elements may implement any of the techniques of like-named elements herein, mutatis mutandis.

For some applications, insertion area 540 of upper layer 530 defines a medical-device penetration region 528 designated for passage of percutaneous medical device 22 through insertion area 540. For some applications, medical-device penetration region 528 comprises a seal 538, which has properties (e.g., material properties or thickness) different in at least one respect from those of material of a remainder of insertion area 540 of upper layer 530. Seal 538 is configured to form a tight seal around percutaneous medical device 22 when device 22 is passed through medical-device penetration region 528. For example, seal 538 may comprise a polymer (e.g., an elastomer), such as silicone, rubber, or synthetic material (e.g., silicone rubber synthetic material), in which case seal 538 may be a septum seal, as known in the drug delivery art. Alternatively or additionally, for example, seal 538 may have a thickness greater than a thickness of the material of a remainder of insertion area 540 of upper layer 530, in which case seal 538 may comprise the same as, or a different material from, the material of the remainder of insertion area 540 of upper layer 530. Using a thinner and/or more flexible material for the remainder of insertion area 540 of upper layer 530 may help ensure that the insertion area as a whole remains highly flexible.

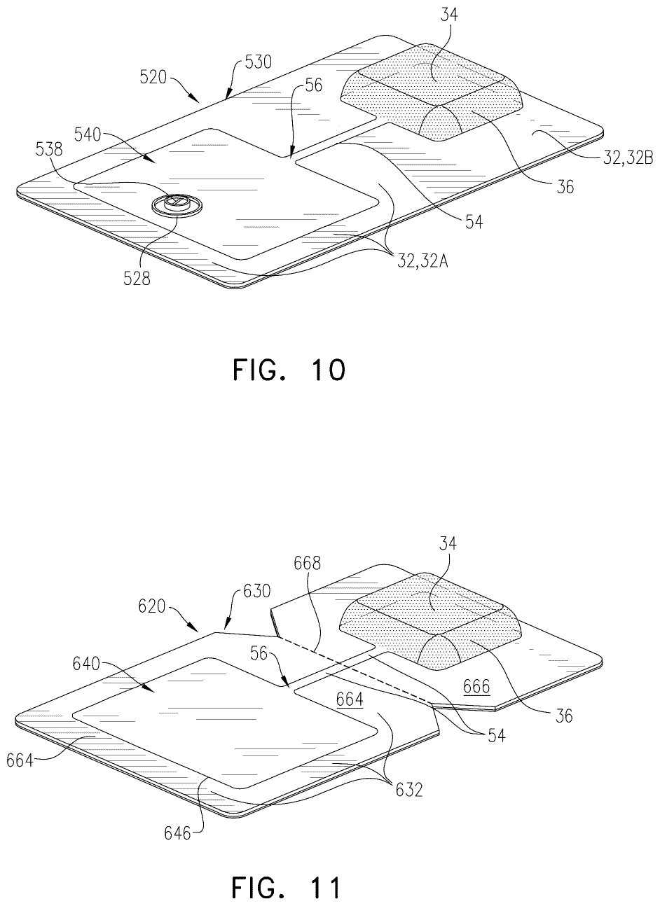

Reference is now made to FIG. 11, which is a schematic isometric view of a dressing 620 for covering a wound of an area of skin of a patient, in accordance with an application of the present invention. Dressing 620 may implement any of the techniques of the other dressings described hereinabove, mutatis mutandis. Like reference numerals refer to like parts.

Dressing 620 comprises an upper layer 630, an adhesive area 632, flowable substance 34, and one or more reservoirs 36 containing flowable substance 34. Upper layer 630 comprises a thin sheet of material, such as a polyurethane film substrate. Upper layer 630 defines a wound-covering area 640 having a non-adhesive skin-facing surface 42 (not shown in FIG. 11, but shown in FIG. 2C for the configuration thereof), configured to be positioned above the wound, in order to protect the wound and surrounding skin area.

Typically, upper surface 44 (not shown in FIG. 11, but shown in FIG. 2C for the configuration thereof) of upper layer 630 is non-adhesive. Optionally, dressing 620 comprises one or more additional layers over upper layer 630 (configuration not shown). Wound-covering area 640 is typically defined by a central area of upper layer 630.

As described above, for some applications, flowable substance 34 comprises an antimicrobial agent (e.g., an antibacterial), an antiseptic agent, and/or a material for treatment of a burn. For other applications, flowable substance 34 comprises a glue, which optionally comprises an antimicrobial agent and/or an antiseptic agent, disposed in the glue. The use of glue, with or without an antimicrobial agent and/or an antiseptic agent, may be appropriate for treating clean cuts through the skin.

For some applications, upper layer 630 defines, in addition to wound-covering area 640, a peripheral area 664, which surrounds at least 50% (e.g., at least 70%, 80%, 90%, or 95%) of a perimeter 646 of wound-covering area 640. For some of these applications, dressing 620 further comprises a reservoir-support layer 666 to which the one or more reservoirs 36 are coupled. Dressing 620 defines a predetermined separation border 668 between peripheral area 664 of upper layer 630 and reservoir-support layer 666. (Predetermined separation border 668 may, for example, be defined by upper layer 630 and/or a peripheral reinforcement, described hereinabove.) For example, predetermined separation border 668 may be defined by scoring, perforation, and/or an easy tear line.

Optionally, predetermined separation border 668 crosses the one or more passages 54, such that the one or more passages 54 are severed when the one or more reservoirs 36 are removed from wound-covering area 640 by separating reservoir-support layer 666 from upper layer 630 at predetermined separation border 668. As mentioned above, the one or more passages 54 may comprise respective valves, such as one-way valves, e.g., duckbill valves, which may prevent leakage of flowable substance 34. Alternatively, leakage may be minimal even if valves are not provided, such as in configurations in which flowable substance 34 is viscous or comprises a glue, and even in configurations in which flowable substance 34 comprises a non-viscous liquid.

During use of dressing 620, after pressure is applied to the one or more reservoirs 36, the one or more reservoirs 36 are removed from wound-covering area 640 by separating reservoir-support layer 666 from upper layer 630 at predetermined separation border 668.

Adhesive area 632 has a skin-facing surface 48 coated with adhesive 50 (not shown in FIG. 11, but shown in FIG. 2C for the configuration thereof). A peripheral portion of adhesive area 632 is disposed around at least a portion of perimeter 646 of wound-covering area 640 (labeled in FIG. 1A), such as around at least 70%, at least 90%, or at least 95% of perimeter 646 of wound-covering area 640. For some applications, adhesive area 632 entirely surrounds perimeter 46 of wound-covering area 640.

For some applications, a skin-facing surface of reservoir-support layer 666 is not coated with an adhesive, thereby facilitating easy removal of the one or more reservoirs 36 during separation of reservoir-support layer 666 from upper layer 630 at predetermined separation border 668. In these applications, a portion of reservoir-support layer 666 may define a skin-facing wall of the one or more reservoirs 36.

For other applications (configuration not shown), adhesive area 632 is a first adhesive area, and dressing 620 comprises a second adhesive area that is configured to adhere the one or more reservoirs 36 to skin 24, such as described hereinabove regarding dressing 20 with reference to FIGS. 1A-B.

Optionally, any of the dressings described herein implement predetermined separation border 668 and some or all of the features thereof.

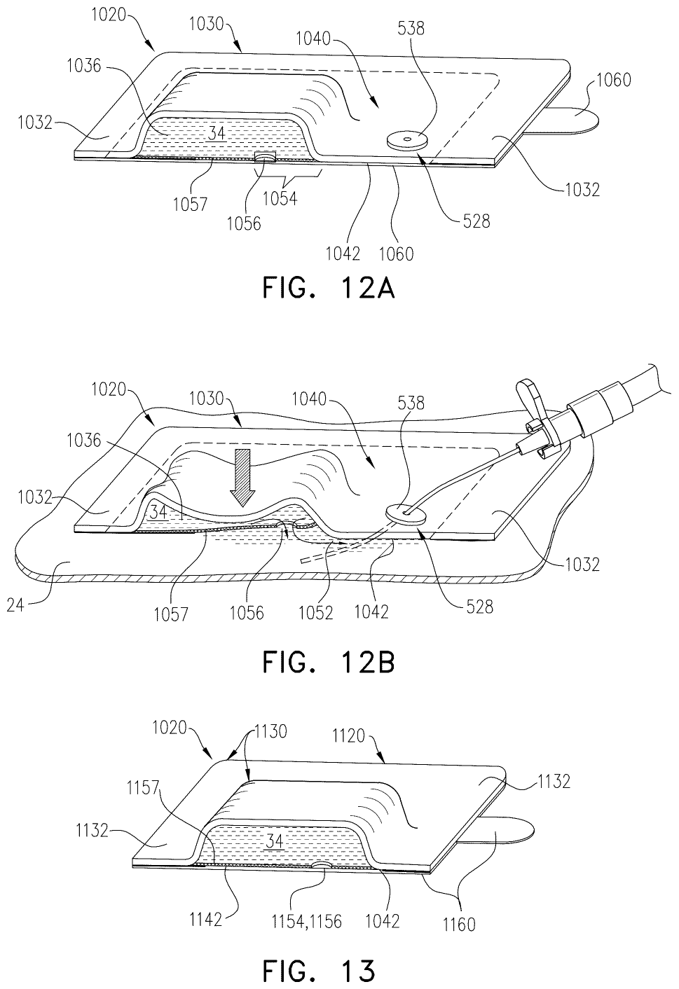

Reference is now made to FIG. 12A, which is a schematic cross-sectional view of a dressing 1020, in accordance with an application of the present invention. Reference is also made to FIG. 12B, which is a schematic illustration of a method for applying dressing 1020 to skin 24, in accordance with an application of the present invention. Other than as described below, dressing 1020 is identical to dressing 20, described hereinabove with reference to FIGS. 1A-2E, and may implement any of the techniques of dressing 20 described hereinabove and/or the other dressings described herein, mutatis mutandis. Likewise, dressing 20 and the other dressings described herein may optionally implement any of the techniques of dressing 1020, mutatis mutandis. Like reference numerals refer to like parts.

Dressing 1020 is for use with percutaneous medical device 22 for insertion through skin 24 of a patient at skin-insertion site 26, as described hereinabove for dressing 20 with reference to FIGS. 2A-E, mutatis mutandis.

Dressing 1020 comprises an upper layer 1030, which defines an insertion area 1040 having a non-adhesive skin-facing surface 1042 configured to be positioned above skin-insertion site 26; these elements may implement any of the techniques of similarly-named elements herein, mutatis mutandis. Dressing 1020 also comprises an adhesive area 1032, flowable substance 34, and one or more reservoirs 1036 containing flowable substance 34 (e.g., exactly one reservoir 1036, such as shown in FIGS. 12A-B).

Insertion area 1040 of upper layer 1030 is shaped so as to define a fillable insertion chamber 1052 between non-adhesive skin-facing surface 1042 and skin 24 when dressing 1020 is adhered to skin 24 by at least a portion of adhesive area 1032, such as shown in FIG. 12B.

For some applications, insertion area 1040 of upper layer 1030 defines medical-device penetration region 528 designated for passage of percutaneous medical device 22 through insertion area 1040, such as described hereinabove with reference to FIG. 10. For some applications, medical-device penetration region 528 comprises seal 538, which has properties (e.g., material properties or thickness) different in at least one respect from those of material of a remainder of insertion area 1040 of upper layer 1030, such as described hereinabove with reference to FIG. 10. Alternatively, insertion area 1040 does not define medical-device penetration region 528, such as described hereinabove for dressing 20 with reference to FIGS. 1A-2E, and for dressing 120 with reference to FIGS. 3A-B.

The one or more reservoirs 1036 are separate from fillable insertion chamber 1052, and are connected to insertion area 1040 by one or more passages 1054 (e.g., exactly one passage 1054) so as to enable flow of flowable substance 34 from the one or more reservoirs 1036 to insertion area 1040 (and fillable insertion chamber 1052) upon application of pressure to the one or more reservoirs 1036.

In this configuration, the one or more reservoirs 1036 are disposed to the side of insertion area 1040, such as shown. The one or more passages 1054 comprise:
a vertical component defined by an opening 1056 through a bottom wall 1057 of the one or more reservoirs 1036; and
a horizontal component 1058 between opening 1056 and at least one edge of insertion area 1040; horizontal component 1058 is bordered on its top by bottom wall 1057 of the one or more reservoirs 1036 and on its bottom by skin 24 when dressing 1020 is applied to the skin.

Reference is made to FIGS. 1A-12B. For any of the applications described herein with reference to FIGS. 1A-12B, percutaneous medical device 22 may comprise a percutaneous-insertion instrument, which may, for example, comprise a tube (e.g., a catheter or a cannula), a cable, a wire, or any other instrument percutaneously inserted into a patient. Although the percutaneous-insertion instrument is illustrated as a peripheral IV catheter in FIGS. 2A-E, the percutaneous-insertion instrument may comprise another type of catheter, such as a peripherally inserted central catheter (PICC), a central catheter, a dialysis catheter, a midline catheter, a urinary catheter, an arterial catheter, a femoral catheter, or an IABP (intra-aortic balloon pump). Exemplary tubes include feeding tubes, external gastric or duodenal tubes, and bile duct tubes. Exemplary cannulas include ECMO cannulas or temporary or longer-term assist devices cannulas. Alternatively, percutaneous medical device 22 may comprise an orthopedic percutaneous fixation device (including an external fixation device), such as a percutaneous pin, or any other device that penetrates the skin, such as an orthopedic percutaneous-insertion instrument.

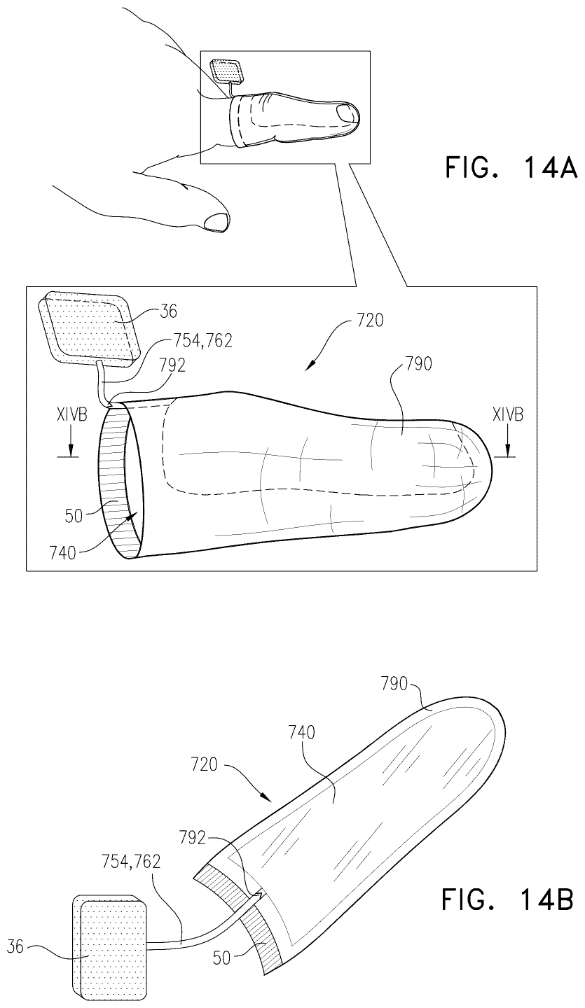

Reference is now made to FIG. 13, which is a schematic cross-sectional view of a dressing 1120, in accordance with an application of the present invention. Other than as described below, dressing 1120 is similar to dressing 20, described hereinabove with reference to FIGS. 1A-2E, and may implement any of the techniques of dressing 20 described hereinabove and/or the other dressings described herein, mutatis mutandis. Likewise, dressing 20 and the other dressings described herein may optionally implement any of the techniques of dressing 1120, mutatis mutandis. Like reference numerals refer to like parts.

Unlike the dressings described hereinabove, dressing 1120 is typically not for use with percutaneous medical device 22, and thus does not comprise an insertion area or a fillable insertion chamber.

Dressing 1120 comprises an upper layer 1130, an adhesive area 1132, flowable substance 34, and one or more reservoirs 1136 containing flowable substance 34 (e.g., exactly one reservoir 1136, such as shown in FIG. 13). A bottom wall 1157 of the one or more reservoirs 1136 has a non-adhesive skin-facing surface 1142 configured to be positioned above skin 24, so as to define a fillable chamber between non-adhesive skin-facing surface 1142 and skin 24 when dressing 1120 is adhered to skin 24 by at least a portion of adhesive area 1132.

The one or more reservoirs 1136 are separate from the fillable chamber, and are connected to the fillable chamber by one or more passages 1154 (e.g., exactly one passage 1154) so as to enable flow of flowable substance 34 from the one or more reservoirs 1136 to the fillable chamber upon application of pressure to the one or more reservoirs 1136.

In this configuration, the one or more reservoirs 1136 are disposed above the fillable chamber, and the one or more passages 1154 (e.g., exactly one passage 1154) comprise one or more openings 1156 (e.g., exactly one opening 1156) arranged vertically between the one or more reservoirs 1136 and the fillable chamber. Alternatively, the one or more reservoirs 1136 are disposed to the side of the fillable chamber (configuration not shown, but similar to the configuration of dressing 20, described hereinabove with reference to FIGS. 1A-2E). Further alternatively, the one or more reservoirs 1136 are disposed partially above and partially to the side of the fillable chamber (configuration not shown, but similar to the configuration of dressing 1020, described hereinabove with reference to FIGS. 12A-B).

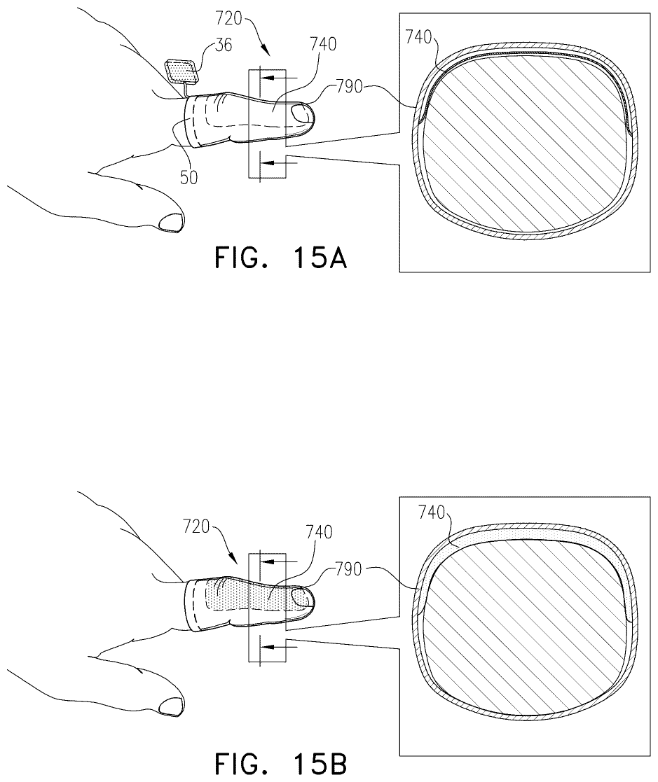

Reference is now made to FIGS. 14A-B, which are schematic isometric and cross-sectional views of a dressing 720 for covering a wound of an area of skin of a finger of a patient, in accordance with an application of the present invention. Dressing 720 may implement any of the techniques of dressing 620, described hereinabove with reference to FIG. 11 and/or the other dressings described hereinabove, mutatis mutandis. Like reference numerals refer to like parts.

Reference is also made to FIGS. 15A-B, which are schematic illustrations of dressing 720 applied to the finger of the patient, in accordance with an application of the present invention.

Dressing 720 is shaped as a finger cot 790. In this configuration, one or more reservoirs 736 are separate from fillable insertion chamber 52, and are connected to wound-covering area 740 by one or more passages 754 comprising one or more respective tubes 762, so as to enable flow of flowable substance 34 from the one or more reservoirs 736 to wound-covering area 740 (and a fillable chamber) upon application of pressure to the one or more reservoirs 736, as shown in the transition between FIG. 15A and FIG. 15B.

Optionally, the one or more passages 754 comprise respective valves 792, such as one-way valves, e.g., duckbill valves, which may facilitate separation of the one or more reservoirs 736 from fillable insertion chamber 52 after pressure is applied to the one or more reservoirs 36.

In an embodiment, techniques and apparatus described herein are combined with techniques and apparatus described in U.S. Provisional Application 63/391,134, filed Jul. 21, 2022, and/or U.S. Provisional Application 63/417,390, filed Oct. 19, 2022, both of which are assigned to the assignee of the present application and incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1.-37. (canceled)

38. A dressing for use with a percutaneous medical device for insertion through skin of a patient at a skin-insertion site, the dressing comprising:
an upper layer, which (a) defines an insertion area having a non-adhesive skin-facing surface configured to be positioned above the skin-insertion site, and (b) is configured to allow passage of the percutaneous medical device through the insertion area;
an adhesive area, which is disposed around at least a portion of a perimeter of the insertion area, and which has a skin-facing surface coated with an adhesive,
wherein the insertion area is shaped so as to define a fillable insertion chamber between the non-adhesive skin-facing surface and the skin when the dressing is adhered to the skin by at least a portion of the adhesive area;
a flowable substance;
one or more reservoirs, which are separate from the fillable insertion chamber, and contain the flowable substance; and
one or more passages connecting the one or more reservoirs of the flowable substance and the insertion area so as to enable flow of the flowable substance from the one or more reservoirs to the insertion area upon application of pressure to the one or more reservoirs.

39.-41. (canceled)

42. The dressing according to claim 38, wherein the flowable substance comprises a substance selected from the group consisting of: a gel, a cream, and a liquid.

43-47. (canceled)

48. The dressing according to claim 38, wherein the adhesive area entirely surrounds the perimeter of the insertion area except at one or more locations at which the one or more passages connect to the insertion area, and wherein the upper layer is configured to allow passage of the percutaneous medical device through the insertion area by puncturing of the upper layer.

49. The dressing according to claim 38, wherein the adhesive area is a first adhesive area, and wherein the dressing comprises a second adhesive area that is configured to adhere the one or more reservoirs to the skin.

50.-54. (canceled)

55. The dressing according to claim 38,
wherein the insertion area of the upper layer is shaped so as to define a slit having first and second edges,
wherein an adhesive-area slit-closing portion of the adhesive area is disposed alongside at least the first edge of the slit, and is configured to be adhered to an upper surface of an upper-layer slit-closing portion of the upper layer alongside the second edge of the slit, and
wherein the upper layer is configured to allow passage of the percutaneous medical device through the insertion area via an opening through the insertion area defined at a junction of respective inner ends of the first and the second edges of the slit, respectively.

56. The dressing according to claim 55, wherein the dressing is shaped such that adhering of the adhesive-area slit-closing portion of the adhesive area to the upper surface of the upper-layer slit-closing portion of the upper layer forms the fillable insertion chamber.

57. The dressing according to claim 56, wherein the dressing is shaped such that adhering of the adhesive-area slit-closing portion of the adhesive area to the upper surface of the upper-layer slit-closing portion of the upper layer provides a tented shape to the fillable insertion chamber.

58. The dressing according to claim 56, wherein the slit is wedge-shaped.

59.-61. (canceled)

62. The dressing according to claim 38, wherein the flowable substance comprises one or more agents selected from the group of agents consisting of: an antimicrobial agent and an antiseptic agent.

63. (canceled)

64. The dressing according to claim 38, wherein the flowable substance comprises a glue.

65. (canceled)

66. The dressing according to claim 38, wherein the flowable substance comprises a liquid hardener of an epoxy, and wherein a solid resin of the epoxy coats the non-adhesive skin-facing surface of the insertion area.

67. (canceled)

68. The dressing according to claim 38, wherein the flowable substance comprises a liquid resin of an epoxy, and wherein a solid hardener of the epoxy coats the non-adhesive skin-facing surface of the insertion area.

69. (canceled)

70. The dressing according to claim 38, wherein the percutaneous medical device is a percutaneous-insertion instrument, and wherein the upper layer is configured to allow passage of the percutaneous-insertion instrument through the insertion area.

71. The dressing according to claim 70, wherein the percutaneous-insertion instrument is a tube selected from the group of tubes consisting of: a catheter and a cannula, and wherein the upper layer is configured to allow passage of the tube through the insertion area.

72. (canceled)

73. The dressing according to claim 70, wherein the percutaneous-insertion instrument is selected from the group of instruments consisting of: a cable and a wire, and wherein the upper layer is configured to allow passage of the selected instrument through the insertion area.

74. (canceled)

75. The dressing according to claim 38, wherein the adhesive area is disposed around at least 70% of the perimeter of the insertion area.

76. (canceled)

77. The dressing according to claim 75, wherein the adhesive area entirely surrounds the perimeter of the insertion area, and wherein the upper layer is configured to allow passage of the percutaneous medical device through the insertion area by puncturing of the upper layer.

78. The dressing according to claim 38, wherein the one or more passages are connected to the insertion area via one or more openings through the insertion area of the upper layer.

79.-80. (canceled)

81. The dressing according to claim 38, wherein the upper layer defines, in addition to the insertion area, a peripheral area, which surrounds at least 50% of the perimeter of the insertion area, and wherein the peripheral area defines the skin-facing surface coated with the adhesive.

82. (canceled)

83. The dressing according to claim 81, further comprising a reservoir-support layer to which the one or more reservoirs are coupled, wherein the dressing defines a predetermined separation border between the peripheral area of the upper layer and the reservoir-support layer, wherein the predetermined separation border is defined by scoring, perforation, or an easy tear line.

84.-89. (canceled)

90. The dressing according to claim 38, wherein, when the adhesive area is adhered to the skin, the one or more passages are open to the skin such that the skin partially defines the one or more passages.

91. The dressing according to claim 90, further comprising a removable protective backing that removably covers (a) the non-adhesive skin-facing surface of the insertion area, (b) the adhesive area, and (c) at least a portion of the one or more passages, so as to seal the one or more passages.

92.-96. (canceled)

97. The dressing according to claim 38, wherein the dressing comprises first and second reservoirs, which contain respective first and second portions of the flowable substance.

98.-104. (canceled)

105. A kit comprising the dressing according to claim 38, the kit further comprising the percutaneous medical device.

106.-110. (canceled)

111. A method comprising:
applying a dressing to skin of a patient by:
positioning, above a skin-insertion site, a non-adhesive skin-facing surface of an insertion area of an upper layer of the dressing, and
adhering at least a portion of a skin-facing surface of an adhesive area of the dressing to the skin, such that the insertion area is shaped so as to define a fillable insertion chamber between the non-adhesive skin-facing surface and the skin, wherein the adhesive area is disposed around at least a portion of a perimeter of the insertion area and is coated with an adhesive;
causing a percutaneous medical device to pass through the insertion area;
before or after causing the percutaneous medical device to pass through the insertion area, passing the percutaneous medical device through the skin at the skin-insertion site; and
after applying the dressing to the skin, applying pressure to one or more reservoirs of the dressing that are separate from the fillable insertion chamber, such that a flowable substance contained within the one or more reservoirs flows from the one or more reservoirs to the fillable insertion chamber via one or more passages connecting the one or more reservoirs and the insertion area.

112. A dressing for covering an area of skin of a patient, the dressing comprising:
an upper layer, which defines a central area having a non-adhesive skin-facing surface configured to be positioned above the skin area;
an adhesive area, which is disposed around at least a portion of a perimeter of the central area, and which has a skin-facing surface coated with an adhesive,
wherein the central area is shaped so as to define a fillable chamber between the non-adhesive skin-facing surface and the skin when the dressing is adhered to the skin by at least a portion of the adhesive area;
a flowable substance;
one or more reservoirs, which are separate from the fillable chamber, and contain the flowable substance; and
one or more passages connecting the one or more reservoirs of the flowable substance and the central area so as to enable flow of the flowable substance from the one or more reservoirs to the central area upon application of pressure to the one or more reservoirs.

113. (canceled)

114. The method according to claim 111,
wherein the insertion area of the upper layer is shaped so as to define a slit having first and second edges, and an adhesive-area slit-closing portion of the adhesive area is disposed alongside at least the first edge of the slit,
wherein causing the percutaneous medical device to pass through the insertion area comprises causing the percutaneous medical device to pass through the insertion area via an opening through the insertion area defined at a junction of respective inner ends of the first and the second edges of the slit, respectively, and
wherein applying the dressing comprises adhering the adhesive-area slit-closing portion of the adhesive area to an upper surface of an upper-layer slit-closing portion of the upper layer alongside the second edge of the slit.

115. The method according to claim 114, wherein causing the percutaneous medical device to pass through the insertion area via the opening comprises, after passing the percutaneous medical device through the skin at the skin-insertion site, positioning the first and the second edges of the slit around a longitudinal portion of the percutaneous medical device such that the percutaneous medical device passes through the opening.

116. The method according to claim 114, wherein adhering the adhesive-area slit-closing portion of the adhesive area to the upper surface of the upper-layer slit-closing portion of the upper layer forms the fillable insertion chamber.

117. The method according to claim 116, wherein adhering the adhesive-area slit-closing portion of the adhesive area to the upper surface of the upper-layer slit-closing portion of the upper layer provides a tented shape to the fillable insertion chamber.
